(19) United States
(12) Patent Application Publication
(10) Pub. No.: US 2021/0338098 A1
(43) Pub. Date: Nov. 4, 2021

(54) PERMANENT MAGNET ROTOR FOR MEDICAL DEVICE TRACKING

(71) Applicant: Lucent Medical Systems, Inc., Kirkland, WA (US)

(72) Inventor: Samuel Peter Andreason, Kirkland, WA (US)

(21) Appl. No.: 16/864,112

(22) Filed: Apr. 30, 2020

(51) Int. Cl. A61B 5/06; G01R 33/44; G01R 33/02; G01R 33/561; G01R 33/28; A61B 34/20

(52) U.S. Cl. CPC A61B 5/062; G01R 33/445; G01R 33/0206; A61B 2034/2051; G01R 33/287; A61B 34/20; G01R 33/5611

(57) ABSTRACT

A medical tracking system including a medical trackable structure configured to be inserted in a body of a patient, a control circuit, and a sensor is provided. The medical trackable structure includes a permanent magnet, and a coil arranged adjacent to the permanent magnet. The control circuit is configured to apply an excitation signal to the coil and rotate the permanent magnet. The permanent magnet is configured to generate a magnetic field including harmonics during the rotation, based in part on the excitation signal applied to the coil. The sensor is configured to sense the harmonics included in the magnetic field and to output to the control circuit a sensor signal based on the magnetic field. The control circuit further calculates position information associated with the medical trackable structure within the body of the patient based on the sensor signal.

### BACKGROUND

### Technical Field

The present disclosure is directed to tracking a magnetic structure within a body. More particularly, but not exclusively, the present disclosure relates to tracking, in real time, a rotating permanent magnet structure stimulated with a low-frequency when the structure is within a body.

### Description of the Related Art

In many medical procedures, a medical practitioner accesses an internal cavity of a patient using a medical tracker or a tracking device. In some cases, the medical practitioner accesses the internal cavity for diagnostic purposes. In other cases, the practitioner accesses the cavity to provide treatment. In still other cases, different therapy is provided.

Due to the sensitivity of internal tissues of a patient's body, incorrectly positioning the medical tracker within the body can cause great harm. Accordingly, it is beneficial to be able to precisely track the position of the medical tracker within the patient's body. However, accurately tracking the position of the medical tracker within the body can be quite difficult. The difficulties are amplified when the medical tracker is placed deep within the body of a large patient.

All of the subject matter discussed in the Description of the Related Art section is not necessarily prior art and should not be assumed to be prior art merely as a result of its discussion in the Description of the Related Art section. Along these lines, any recognition of problems in the prior art discussed in the Description of the Related Art section or associated with such subject matter should not be treated as prior art unless expressly stated to be prior art. Instead, the discussion of any subject matter in the Description of the Related Art section should be treated as part of the inventor's approach to the particular problem, which in and of itself may also be inventive.

### BRIEF SUMMARY

The present disclosure provides a rotating magnetic structure in a body of patient during a medical procedure and method of tracking the same. The method can determine the location and the motion of the rotating magnetic structure with 6 DOF (degree of freedom) by analyzing the magnetic field generated due to the rotating magnetic structure. The magnetic structure may include a permanent magnet. The magnetic structure is configured to be inserted into a body of a patient during a medical procedure and a sensor located outside of the body may be able to track the location of the magnetic structure with a high level of precision. The magnetic structure may also be incorporated as part of a medical or surgical instrument to provide for knowing the exact location of the medical instrument during a medical procedure.

The rotating magnetic structure according to the present disclosure can be within a probe that has an overall form factor that is small enough to be inserted or planted within a number of different locations in the body of the patient. In some embodiments, the ratio between the length L and the diameter D of the magnetic structure (L/D) may be close to 1, namely, the entire probe may have a length and diameter that are approximately equal to each other. This permits the probe to be used in very confined locations in the body of a patient, for example, within the spinal cord or positioned adjacent to a vertebrae of a patient during a back surgery to assist the medical practitioners in knowing the exact location of their medical instruments during back surgery.

In addition, the rotating magnetic structure according to the present disclosure may be operated based on a low frequency excitation signal. The magnetic structure is possible to be detected at a frequency as low as 20 Hz to as high as 2500 Hz. The rotating magnetic structure does not causes excess heat to make the patient uncomfortable during the medical procedure.

In some aspects of the present disclosure, the position of the rotating magnetic structure can be located exactly without the use of harmonics associate with the magnetic field. That is, in some embodiments, even without using the harmonics, obtaining either a 5 DOF or 6 DOF information is possible based using the fundamental frequency of the magnetic field and the phase information of the tracked magnetic structure and the sensor tracking the structure.

A further, aspect of the present disclosure provides a medical tracking system. The medical tracking system includes a permanent magnet assembly having a permanent magnet, a conductive wire coil positioned adjacent to the permanent magnet, a control circuit configured to apply an excitation signal to the wire coil and cause rotation of the permanent magnet, and a sensor positioned outside the body of the patient.

In some embodiments, the permanent magnet generates a magnetic field having harmonics in the magnetic field during the rotation. The sensor is configured to sense the harmonics of the magnetic field and to output a sensor signal based on the magnetic field that indicates the location of the permanent magnet assembly within the body of the patient based on the sensor signal.

According to one aspect of the present disclosure, medical trackable apparatus configured to be inserted in a body of a patient is taught. In one embodiment, the medical trackable apparatus includes a fixed shaft, a magnetic structure configured to revolve around the fixed shaft at a first rotation rate, coils spaced apart from the magnetic structure at an outer periphery of a permanent magnet, an air gap between the magnetic structure and the coils, and a first bearing structure directly contacting the fixed shaft. In one embodiment, the bearing rotates with the shaft and as the same speed, the bearing being the contact member between the rotating magnet and the stationary housing. In another embodiment, the bearing will revolve around the shaft at a second rotation rate, the second rotation rate being different from the first rotation rate.

In some embodiments, the coils receive an excitation signal causing the magnetic structure to revolve around the fixed shaft. The revolving magnetic structure generates a plurality of trackable harmonics associated with a magnetic field, when the excitation signal is received. For example, one or more trackable harmonics from the revolving magnetic structure may be used for calculating the location, orientation, etc. of the revolving magnetic structure. Even one trackable harmonic at one frequency of magnetic field can provide viable tracking.

Yet another aspect of the present disclosure is a method to track a low-frequency trackable structure. The method includes: advancing a medical device into a body of a patient, the medical device having a low-frequency trackable structure affixed thereto; applying a low-frequency excitation signal to the low-frequency trackable structure; rotating the low-frequency trackable structure to generate a magnetic field; determining in real time, from outside of the body of the patient, at least one harmonics associated with the magnetic field produced by the low-frequency trackable structure; and presenting visual information that tracks motion of the medical device inside the body of the patient based on the detection of the at least one harmonics associated with the magnetic field.

In still another aspect of the present disclosure, a medical tracking system capable of generating a 5 DOF tracking is also available. In some embodiments, the fundamental frequency data of the magnetic field may be used without harmonic generation or detection to provide a 5 DOF tracking.

In one or more embodiments, the medical tracking system according to the present disclosure may only generate one frequency of the magnetic field to provide viable tracking of the magnetic trackable structure.

Additional aspects of the present disclosure will be described herein.

### BRIEF DESCRIPTION OF THE SEVERAL VIEWS OF THE DRAWINGS

Reference will now be made by way of example to the accompanying drawings. In the drawings, identical reference numbers identify similar elements or acts. In some drawings, however, different reference numbers may be used to indicate the same or similar elements. The sizes and relative positions of elements in the drawings are not necessarily drawn to scale. For example, the shapes of various elements and angles are not necessarily drawn to scale, and some of these elements may be enlarged and positioned to improve drawing legibility:

FIG. 1 illustrates a view of a medical tracking device, in accordance with one or more embodiments of the present disclosure;

FIG. 2A is a perspective view of a magnetic structure in a trackable structure used for detecting the position of the trackable structure within a body of a patient, according to one or more embodiments;

FIG. 2B is a cross-sectional view of a trackable structure, according to one embodiment of the present disclosure;

FIG. 2C is a cross-sectional view of a trackable structure, according to another embodiment of the present disclosure;

FIG. 2D is a cross-sectional view of a trackable structure, according to another embodiment of the present disclosure;

FIG. 2E is a cross-sectional view of a trackable structure taken along section line E-E′ of FIG. 2B, according to one embodiment of the present disclosure;

FIG. 2F is a top view of coils of a trackable structure according to one embodiment of the present disclosure;

FIG. 2G is a top view of coils of a trackable structure according to another embodiment of the present disclosure;

FIG. 2H is another top view showing the configurations of the coils of a trackable structure shown in FIG. 2F;

FIG. 3 is a block diagram of a medical tracking system for detecting the position of a trackable structure within a body of a patient, according to one or more embodiments;

FIG. 4A is a cross-sectional view of a trackable structure and the magnetic field lines, according to one or more embodiments;

FIGS. 4B, 4C, 4D are cross-sectional views of various embodiments of a trackable structure showing the relationship between the magnetic field lines generated from a magnetic structure with respect to the axis of spin and the arrangements of the coils;

FIGS. 4E, 4F, 4G are graphs of magnetic fields of a trackable structure collected from sensors at various locations, according to one or more embodiments;

FIG. 4H is a graph showing harmonics included in the magnetic fields generated by a trackable structure as detected by a sensor at a first location, according to one or more embodiments;

FIG. 4I is a graph showing harmonics included in the magnetic fields generated by a trackable structure as detected by a sensor at a second location, according to one or more embodiments;

FIG. 4J is a graph showing various harmonics of various frequencies with different phase and amplitude information;

FIG. 5 illustrates a sensor, according to one or more embodiments;

FIG. 6A illustrates a low-frequency medical tracking system for detecting a position of a trackable structure within a body of a patient, according to at least one embodiment;

FIG. 6B illustrates a low-frequency medical tracking system for detecting a position of a trackable structure within a body of a patient, according to at least one embodiment;

FIG. 7A is a graph of a square wave excitation signal that can be applied to a coil of the trackable structure, according to one or more embodiments;

FIG. 7B is a graph of a sine wave excitation signal that can be applied to a coil of the trackable structure, according to one or more embodiments;

FIG. 8 is a flowchart of a method for detecting the position of a trackable structure within a body of a patient, according to one or more embodiments of the present disclosure;

FIG. 9A is a diagram illustrating a rotating trackable structure, according to one or more embodiments;

FIG. 9B is a diagram illustrating a rotating trackable structure, according to another embodiment of the present disclosure;

FIG. 10A illustrates a perspective view of a modified housing of a trackable structure according to one embodiment of the present disclosure;

FIG. 10B illustrates a front view of the modified housing of the trackable structure of FIG. 10A;

FIG. 10C illustrates a side view of the modified housing of the trackable structure of FIG. 10A;

FIG. 11A illustrates a perspective view of a modified housing of a trackable structure according to another embodiment of the present disclosure;

FIG. 11B illustrates a front view of the modified housing of the trackable structure of FIG. 11A;

FIG. 11C illustrates a side view of the modified housing of the trackable structure of FIG. 11A;

FIG. 12A illustrates a perspective view of a secondary housing for housing a trackable structure according to one embodiment of the present disclosure;

FIG. 12B illustrates a front view of the secondary housing for housing the trackable structure of FIG. 12A; and

FIG. 12C illustrates a cross-sectional view of the secondary housing for housing the trackable structure of FIG. 12A.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures or methods associated with magnetic trackable structures have not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

A medical tracking device having a new trackable structure is contemplated. The trackable structure includes a low-frequency electromagnetic apparatus that is trackable with a magnetic field sensing device. The magnetic field sensing device includes particular algorithms to identify and track the position of the low-frequency permanent magnet apparatus in three dimensions and the orientation of low-frequency permanent magnet apparatus relative to a reference point. A display associated with the magnetic field sensing device presents output information to a medical practitioner representing the position and orientation of at least one of the trackable structure and the low-frequency permanent magnet apparatus.

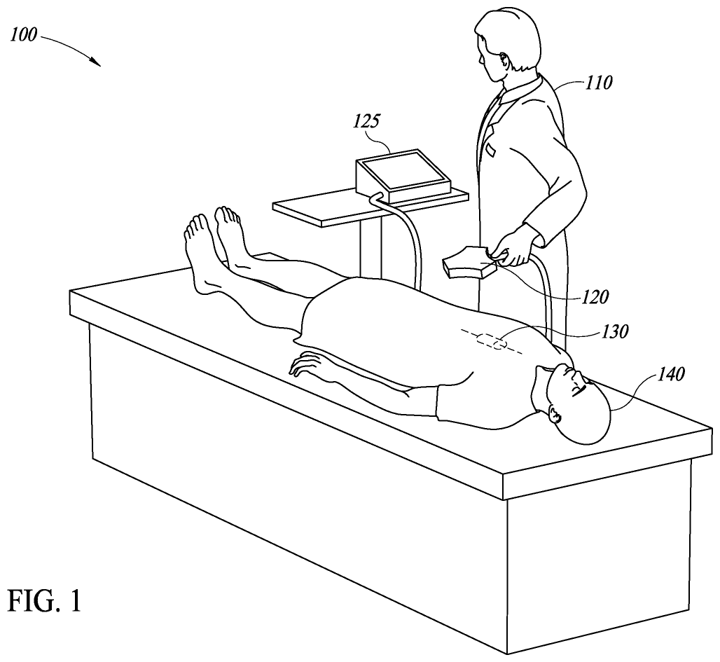

FIG. 1 illustrates a view of a medical tracking device 100, in accordance with one or more embodiments of the present disclosure. The figure illustrates a clinical setting (e.g., spine surgery) in which a clinician or a medical practitioner 110 tracks a location of a trackable structure 130 (also referred to as a motor assembly 130 in some embodiments) inserted inside a patient 140 using the medical tracking device 100. In one or more embodiments, the medical tracking device 100 includes the trackable structure 130, a sensor 120, and a monitoring station 125. This is not an exhaustive list of the components that may be included in medical tracking device 100 and other components may be incorporated as needed.

The sensor 120 used by the medical practitioner 110 (including doctors, nurses, and others) receives the locations of the trackable structure 130. The trackable structure 130 is a motor is in the class of permanent magnet motors in which the center rotating member 230 that is the rotor is comprised of a permanent magnet. The sensor 120 is electrically connected to the monitoring station 125 so that the locations of the trackable structure 130 is shown on the display screen of the monitoring station 125. Multiple trackable structures can be positioned within the various body parts of the patient 140 to assist the medical practitioner 110 during the medical procedures. For example, one or more trackable structures 130 may be attached to the patient's vertebrae during a spine surgery. By having these trackable structures 130 fixated to the vertebrae of the patient, the medical practitioner 110 may benefit from accurately locating the position of each of the vertebrae and assist him/her during the spine surgery.

The sensor 120 is configured to acquire magnetic fields, electro-magnetic signals, harmonics generated from the magnetic field and the electro-magnetic signals, and any other suitable signals originating from the trackable structure 130 placed within the body parts of the patient 140. These data collected from the sensor 120 may be processed and produced in the form of images so that it can be displayed at the monitoring station 125.

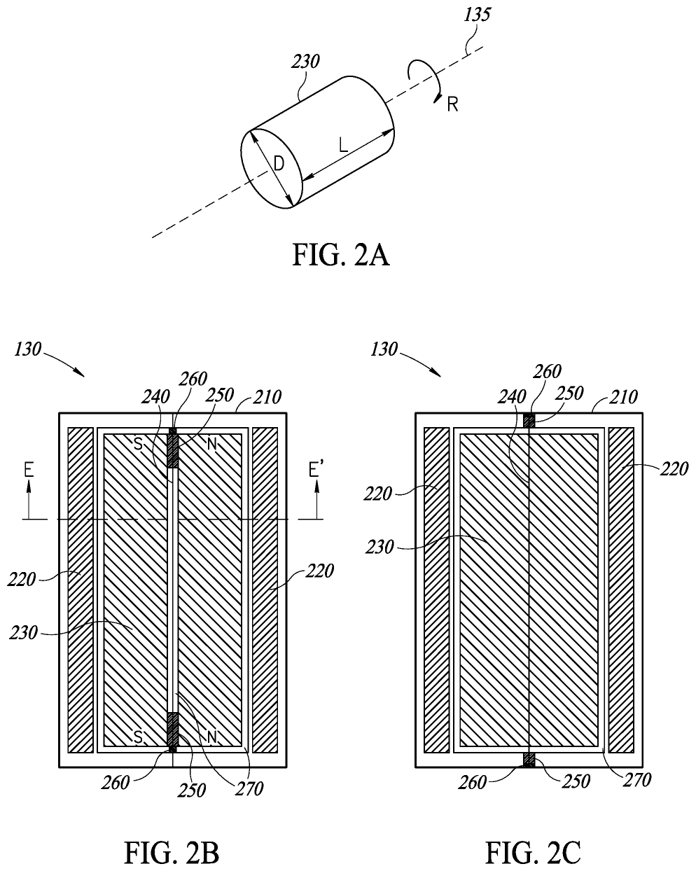

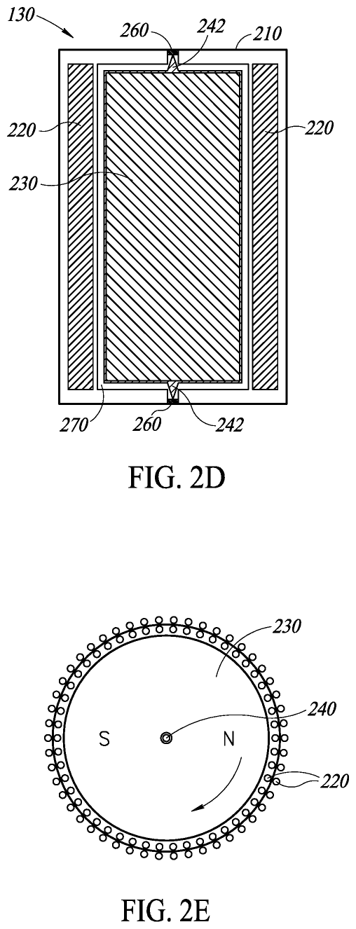

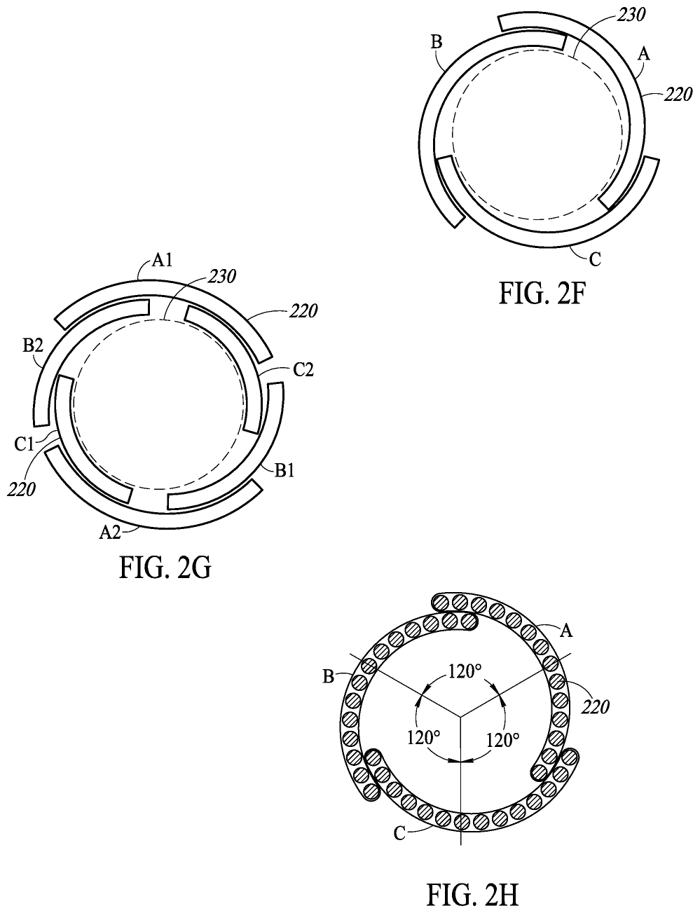

FIG. 2A is a perspective view of a magnetic structure 230 in a trackable structure 130 used for detecting the position within a body of a patient, according to one or more embodiments. The magnetic structure 230 which is a component of the trackable structure 130 rotates along a rotational axis 135 in a direction R. The direction R of the rotation may be clockwise or counter clockwise depending on the specific application. The form factor of the trackable structure 130 may be in the range of 0.5<L/D<5. In some embodiments, the form factor of the trackable structure 130 may be close to or exactly L/D=1, explained later herein. The components and the structural details of the trackable structure 130 will be explained in FIGS. 2B to 2D.

FIG. 2B is a cross-sectional view of a trackable structure 130, according to one embodiment of the present disclosure. The trackable structure 130 is covered by a housing 210. The housing 210 can be made of a material such as a plastic or an epoxy. Further, examples may include, but are not limited to, silicone-based materials, biocompatible polymers, synthetic polymers, resin and the like.

Additionally, other suitable materials may be used for the housing 210. The trackable structure 130 is intended to be inserted inside of the patient and in direct contact with the patient's skin, organs, or other body parts during use of the medical tracking device 100. Accordingly, any biocompatible material which is compatible with living tissue and which does not produce a toxic or immunological response when exposed to the body may be utilized. In some embodiments, the entire housing 210 may be formed of a biocompatible material or parts of the housing 210 of the trackable structure 130 may be partially coated with a biocompatible coating material.

Inside the housing 210 is a coil (a conductive wire coil) or a coil area 220 where the coils are located. The coils 220 are wound at a location adjacent to a magnetic structure 230. In one embodiment, the magnetic structure is a permanent magnet. The coils 220 may be wound along a length direction L (or an axial direction) of the magnetic structure 230. In one or more embodiments, the coils 220 are stationary and are not spinning or rotating. A larger coil 220 will cause a correspondingly larger disruption of tissues or other biological matter that make up the body of the patient 140 as the trackable structure 130 that bears the magnet structure 230 is introduced into the body of the patient 140. Thus, the size of the coil 220 may be selected to be an appropriate dimension to comply with the overall form factor of the trackable structure 130.

One medical application of the form factor of the trackable structure 130 is in surgical settings (e.g., spinal surgery). For example, a medical practitioner may attach the trackable structure 130 into the vertebrae to track the location of the vertebrae. In some cases the medical practitioner may wish to attach multiple trackable structures 130 to track multiple vertebrae simultaneously. The trackable structures 130 will be able to show how the vertebrae are lined up during the spine surgery. Using the trackable structure 130 as a medical implant for tracking spine is one application of the trackable structure 130 according to the present disclosure, and many other applications may be contemplated.

In one or more embodiments, the magnetic structure 230 includes a permanent magnet, however other suitable magnetic structures may be included in other embodiments. A permanent magnet 230 is an object made from a material that is magnetized and creates its own persistent magnetic field. The permanent magnet 230 may include a ferromagnetic material, a paramagnetic material, or another type of material.

Permanent magnet strength depends upon the material used in its creation. With modern high magnetic coercivity materials it is possible to magnetize a cylinder shape permanent magnet 230 in the transverse (to axis of symmetry) direction. In one or more embodiments, the permanent magnet 230 is transverse polarized. As shown, the coils 220 are wound at the periphery of the permanent magnet 230. The coils 220 are being driven at some designed frequency (e.g., frequency based on an excitation signal) which will be detailed later on.

In a preferred embodiment, the permanent magnet rotor 230 is a cylindrical shape. In a preferred embodiment, it is one magnet with two poles, namely a single dipole magnet. The north pole (N pole) makes up a first half of the cylinder and the south pole (S pole) comprised the other half of the cylinder. In FIG. 2A, the N pole is shown on the right hand side, comprising that entire half cylinder from the top to the bottom and S pole is shown on the left hand side, comprising that entire half cylinder from the top to the bottom, as shown. As it rotates, of course, the location of the poles will switch.

FIG. 2A shows the class of rotor being used, namely it is permanent magnetic rotor. There is no central shaft to be driven by the rotor because in this design, the goal is to detect the change in magnetic flux as the magnet cylinder rotates.

The preferred form of this rotor that will provide the strongest signal is to maximize the amount of material that is fixed magnetic with the magnetized direction all in the same direction. The preferred overall structure is that of a dipole in which one entire half of the cylinder is one pole, N and the other entire half of the cylinder is the other pole, S.

There can be a neutral space between the right and left half cylinders of the rotor, but this is not required. For example, another form might be two provide consistently magnetized half cylinders of a permanent magnet with a soft magnetic plate sandwiched in between. This soft magnetic plate in the sandwich would present a rotor phase (orientation) dependent change in inductance for the drive circuit which could result in better phase detection. There are other rotor designs that could be used, but in most circumstances are not preferred because the output signal will be as strong. For example, the rotor could be the type having surface mounted permanent magnets on a metal core that is not a magnet itself. For example, having four such magnets mounted circumferentially around the perimeter, the magnets having two poles each, a north and a south at either end of the magnet. Thus the outer shell of the cylinder could be composed of individual hard magnets with the inside being a magnetic material, such as iron. This might be an approach to obtain a lower cost rotor since having a central cylinder made of metal, but not having magnet center would be lower cost than a more expensive hard magnetic material that makes up the entire cylinder. Alternatively, it could have interior magnets, mounted as embedded or inserted. Such designs could have four, six, eight or more magnets, each with two poles. Thus, the permanent magnet 230 having various forms and shapes may be contemplated. As can be appreciated, a quadrupole, etc. magnet, particularly if the magnet comprises only a portion of the rotor material will result in a signal that will fall off very quickly with distance. Thus there are tradeoffs in cost versus signal strength.

In the preferred embodiment of the entire rotor being comprised of a single dipole permanent magnet material, a very small device and provide a strong signal that is detected outside the body, resulting in significant medical benefits from such a design choice. In the embodiment of FIG. 2B, in the hollow central space 270 within the permanent magnet 230 is a shaft 240 which the permanent magnet 230 revolves around. The shaft 240 is stationary in this design. Between the permanent magnet 230 and the shaft 240 is a first bearing structure 250. The first bearing structure 250 is coupled to the shaft 240 and contacts the inner surface of the permanent magnet 230. The first bearing structure 250, for example, includes a sleeve bearing, a bushing, or the like. These examples are not limited and other suitable bearings may be used. One or more first bearing structures 250 may be used. For example, one sleeve bearing (i.e., a first sleeve bearing) may be located adjacent to a first end of the shaft 240 and another sleeve bearing (i.e., a second sleeve bearing) may be located adjacent to a second end of the shaft 240 that is opposite to the first end. In some embodiments, the first sleeve bearing that is positioned near the first end of the shaft 240 is substantially coplanar with a top surface of the permanent magnet 230 and the second sleeve bearing that is positioned near the second end of the shaft 240 is substantially coplanar with a bottom surface of the permanent magnet 230.

A second bearing structure 260 is located between the housing 210 and the first bearing structure 250. Additionally or alternatively, the second bearing structure 260 is positioned within a space (or an air gap) 270 between the housing 210 and the permanent magnet 230. In one or more embodiments, the second bearing structure 260 is coupled to the shaft 240 and is in contact with the first bearing structure 250 and the housing 210. The second bearing structure 260, for example, includes an axial thrust bearing or a plastic washer, or the like. These examples are not limited and other suitable bearings may be used. One or more second bearing structures 260 may be used. For example, one axial thrust bearing (i.e., a first axial thrust bearing) may be positioned on one end of the first sleeve bearing and another axial thrust bearing (i.e., a second axial thrust bearing) may be positioned on one end of the second sleeve bearing.

In some embodiments, the first bearing structure 250 is mechanically fixed, such as by an adhesive or a press fit to the permanent magnet 230 (e.g., a rotor). For example, the first and second sleeve bearings 250 are mechanically glued to the permanent magnet 230 and they rotate around the fixed shaft 240. In some embodiments, there are additional flat plastic washers 260 or the like on the shaft 240 that may contact the sleeve bearings.

In other embodiments, the shaft 240 could be conductive to help float the permanent magnet 230. Yet in other embodiments, the shaft 240 may be formed non-conductive or semi-conductive to reduce inductive heating.

During operation, that is, when the trackable structure 130 is rotating along the shaft 240, the permanent magnet 230 and the first bearing structure 250 rotate at a first rotation rate. That is, the shaft 240 is fixed and may not spin and the permanent magnet 230 and the first bearing structure 250 may spin. Further, the second bearing structure 260 abuts the first bearing structure 250 but the second bearing structure 260 may not spin. In some embodiments, the second bearing structure 260 rotates at a second rotation rate that is different from the first rotation rate. For example, the second bearing structure 260 is free to rotate at a different rate than every other component within the trackable structure 130. However, in other embodiments, the second bearing structure 260 may rotate at the first rotation rate as the permanent magnet 230 and the first bearing structure 250. The second bearing structure 260 can spin at a different rate as it is not locked to the housing 210 or the shaft 240. It may spin at a different rate than the housing 210 or the shaft 240 because of the difference in frictional torques. For example, the shaft 240 and housing 210 are trying to slow it down while the first bearing structure 250 is trying to speed it up.

In one or more embodiments, the rotational rate may range from about 20 Hz to 2.5 kHz. In some embodiments, it may be preferable to operate between about 200 Hz and 650 Hz. However, various ranges of the rotational rate may be applied based on the clinical setting, the medical procedure, and the medical application. For example, in some cases, the rotation rate of the permanent magnet 230 may be below 20 Hz. While this is theoretically possible to implement, operating at a slower rotation rate may cause the patient and the medical practitioner to wait, for example, about 10 seconds or so to get one updated location of the trackable structure 130. In some embodiments, higher frequencies of operation may be used to efficiently control energy losses, where frequency of operation refers to the rotational rate. Lower energy losses, by lowering frictional losses for example, would enable higher frequencies of operation.

Higher frequencies of operation would permit the use of a larger number of tracked magnetic objects 130 simultaneously in the same patient. Thus, if larger range of frequencies can be used, then many different rotors can be place in the patient, some operating at low frequencies, some a mid-range frequencies and some operating at high frequencies with the range. Thus, the ability to use higher frequencies increases the range and also the number of tracked objects 130 that can used while having a sufficient difference in frequency of operation that each can be detected and tracked.

In one or more embodiments, the permanent magnet 230 may rotate at a constant rotational rate. However, in other embodiments, the permanent magnet 230 may rotate at a non-constant rotational rate. That is, any rotation rate for appropriately operating the trackable structure and the medical tracking system described herein may be used and that rate might vary over time for a single trackable object 130. This would be one way to generate the harmonics for phase detection, to obtain the 6th DOF sensing. Thus, the drive coils might make it so the rotor periodically speeds up and slows down when it nears an alignment orientation.

In one or more instances, various rotor design choices may be implemented because coupling to the rotor or the shaft is not required for the mechanical rotation. For example, an alternate version may glue the shaft 240 to the rotor 230, and remove the second bearing structure 260 (e.g., plastic washer), and seat the shaft 240 into a conical section. This alternate has the potential to reduce frictional losses because of a smaller interaction surface radius. However, in some cases, this may be less capable of taking transverse loadings. In further variations, surfaces could be treated to reduce friction (e.g., sapphire, etc.). Further variations of the trackable structure 130 design will be further explained in connection FIGS. 2C and 2D.

In one or more embodiments, the housing 210 may be sealed and filled with some gases or sealed in a vacuum state or sealed at a suitable pressure for reducing or minimizing dissipation during the rotation of the permanent magnet 230. For example, the interior of the housing 210 may be filled with helium (He) or any other lighter gas. In some embodiments, the space 270 between the coil 220 and the permanent magnet 230 may be filled with light gas such as He or may be kept at a vacuum state or other suitable gases with suitable pressure for reducing the dissipation during rotation may be used. In other embodiments, the space 270 within the hollow space of the donut-like or tube-like cylinder shaped magnet 230 may be filled with suitable gas or kept at a vacuum state.

When the permanent magnet 230 within the trackable structure 130 rotates, the rotation causes a rotating magnetic field. Accordingly, unlike an oscillating magnetic field where there are times when there is no field to be sensed (albeit short), there is always a magnetic field to be sensed in a rotating magnetic field. For example, in an oscillating magnetic field situation, the tracked magnetic dipole (or simply dipole) is always aligned with the (rotation) axis, so it is intrinsically capable of a 5 DOF (degree of freedom) tracking. On the other hand, when there is a rotating magnetic field, the tracked dipole is always transverse to the rotating axis. The rotation is an additional inferred parameter that allows the sensor 120 to accomplish 6 DOF tracking without the use of an additional independent tracked element. Accordingly, the medical tracking device 100 according to the present disclosure is capable of tracking the location of a trackable structure 130 within the patient 140 with a 6 DOF. The method of obtaining the location of a trackable structure 130 with a 6 DOF will be detailed later on.

Alternatively, or in addition, the permanent magnet 230 may be composed of two or more magnet structures rather than a single continuum structure.

FIG. 2C is a cross-sectional view of a trackable structure 130, according to another embodiment of the present disclosure. In this embodiment, the shaft 240 may be glued to a rotor or the magnetic structure 230 (e.g., permanent magnet) and the ends of the shaft 240 could be supported in a needle like arrangement.

Similarly to FIG. 2B, a coil or a coil area 220 is located inside the housing 210. The coils 220 are wound at a location adjacent to the permanent magnet 230. The coils 220 may be wound at the periphery of the permanent magnet 230 along a length direction L (or rotational axis direction) of the permanent magnet.

In this example, as mentioned above, the permanent magnet 230 does not have a hollow space within the permanent magnet 230. That is, rather than having a tube-like cylindrical shape, this example has a solid cylindrical shape where the shaft 240 extends through the rotational axis of the permanent magnet 230 in which the magnet revolves around a needle-like shaft. At one end of the shaft 240 is a second bearing structure 260 and at the other opposite end of the shaft 240 is another second bearing structure 260. These second bearing structures may be identical but located at two different locations of the shaft 240. The first bearing structure 250 is arranged between the second bearing structure 260 and the permanent magnet 230. The housing 210 has a first opening on a top surface of the trackable structure 130 and a second opening on a bottom surface of the trackable structure 130. The first and second bearing structures 250, 260 are positioned on the first and second openings, respectively. The first bearing structure 250 is coupled to the shaft 240 and contacts an inner surface of the housing 210. In some embodiments, the second bearing structure 260 which includes, for example, an axial thrust bearing or a plastic washer may be substantially coplanar with a top surface (e.g., top exterior surface) of the housing 210 and the other second bearing structure 260 that is positioned near the second end of the shaft 240 may be substantially coplanar with a bottom surface (e.g., bottom exterior surface) of the housing 210.

When the trackable structure 130 is rotating along the shaft 240, the permanent magnet 230 and the first bearing structure 250 rotates at a first rotation rate. In some embodiments, the second bearing structure 260 rotates at a second rotation rate that is different from the first rotation rate. For example, the second bearing structure 260 is free to rotate at a different rate than every other component within the trackable structure 130. However, in other embodiments, the second bearing structure 260 may rotate at the first rotation rate as the permanent magnet 230 and the first bearing structure 250.

FIG. 2D is a cross-sectional view of a trackable structure 130, according to another embodiment of the present disclosure. In this embodiment, an internal shaft 240 is not present. Instead, conical bearing 242 may be fixed to at the top and bottom of the magnetic structure 230 (e.g., permanent magnet) and the ends of the shaft 242 may have a conical pointed tip shape for low friction bearing support. That is, the conical tip shaped shaft 242 may be glued to a top and bottom surface of the permanent magnet 230. As shown, in this example, the permanent magnet 230 does not have a hollow space within it and is a cylindrical continuum rather than a tube-like cylindrical shape. It has one half of the cylinder that is a North pole and the other half is the South pole, but these are not marked for ease of understanding the view.

In these embodiments like FIG. 2D, the first bearing structure 250 may not be needed and the presence of the second bearing structure 260 including an axial thrust bearing or a plastic washer may suffice. The second bearing structure 260 includes a first axial thrust bearing that is contacting a conical tip of the shaft at one end and a second axial thrust bearing that is contacting a conical tip of the shaft at the other opposite end.

The housing 210 has a first opening on a top surface of the trackable structure 130 and a second opening on a bottom surface of the trackable structure 130. The first axial thrust bearing is positioned on the first opening and the second axial thrust bearing is positioned on the second opening. In some embodiments, the second bearing structure which includes, the first and second axial thrust bearings are substantially coplanar with a top and bottom surfaces (i.e., exterior top, bottom surfaces) of the housing 210, respectively.

In one or more embodiments, the housing 210 may be sealed and filled with some gases or sealed in a vacuum state or sealed at a suitable pressure for reducing or minimizing dissipation during the rotation of the permanent magnet 230. For example, the interior of the housing 210 may be filled with helium (He) or any other lighter gas. In some embodiments, the space 270 between the coil 220 and the permanent magnet 230 may be filled with light gas such as He or may be kept at a vacuum state or other suitable gases with suitable pressure for reducing the dissipation during rotation may be used.

When the permanent magnet 230 glued with the conical tip shaft 240 is rotating during operation at a first rotation rate, the second bearing structure 260 may be fixed or may rotate at a second rotation rate that is different from the first rotation rate. For example, the second bearing structure 260 can be free to rotate at a different rate than the various components within the trackable structure 130 such as the permanent magnet 230 and the conical tip shaft 240. However, in other embodiments, the second bearing structure 260 may rotate at the first rotation rate as the permanent magnet 230 and the shaft 240.

In some embodiments, the permanent magnet 230 could be machined to have that end shaft type of support. For example, a piece of the shaft 240 may be glued or plated on the permanent magnet. As shown in FIG. 2D, the permanent magnet 230 may be plated (which may be helpful for oxidation resistance) and that plating may serve a purpose of making an axial support shaft.

In further embodiments, additional bearing systems may be applied. For example, the sleeve may be replaced in lieu of the additional bearing systems. Further, spring supported systems may also be implemented so that the spinning part is not out of contact with the various supporting structures.

The trackable structure 130 according to the present disclosure is not limited by balancing magnetic L/D due to self de-magnetizing fields, or against greater magnetic coercivity coupled with higher power requirements. For example, while generally when a dimension of a magnetic structure, in this example, L becomes shorter, the power requirements to drive the rotor may increase, but the trackable structure 130 according to the present disclosure is not constrained in this regard.

FIG. 2E is a cross-sectional view of a trackable structure 130 taken along E-E′ of FIG. 2B, according to one embodiment of the present disclosure. The figure shows that the magnetic structure 230 revolves around the axis of the shaft 240. However, the permanent magnet 230 does not directly contact the shaft 240 and has a tube-like cylindrical shape that has a hollow space within the magnet 230. The general location of the two poles, N and S are also shown in FIG. 2E. Each of the poles occupies one half of the cylinder and as can be appreciated, there will be various field lines of different strengthens and lengths between them and a junction along a diameter line of the cylinder where the north and south meet, but this junction of the north and south poles is not shown. Various arrangements of the coil can be contemplated. FIGS. 2F and 2G illustrate examples of the arrangements of the coil systems.

FIG. 2F is a top view of coils of a trackable structure 130 according to one embodiment of the present disclosure. FIG. 2H is another top view showing the configurations of the coils 220 of a trackable structure shown 130 in FIG. 2F.

In FIG. 2F, the coils can have a 3 phase coil system where a group of coils are grouped as coil group A, coil group B, and coil group C, which have a 120° phase difference from each other. For example, coil group A overlaps with coil group C, and coil group C overlaps with coil group B, and coil group B overlaps with coil group A. In FIG. 2H, each of the coils within the grouped coils or coil groups are shown. The boxed rectangles in FIGS. 2B-2D and curved rectangles in FIGS. 2F and 2G that indicate the coils 220 are drawn in this manner for simplicity purposes, and a person of ordinary skill in the art will readily understand the structures of the coils, the alignments and the arrangements of the coils based on the present disclosure.

FIG. 2G is a top view of coils of a trackable structure 130 according to another embodiment of the present disclosure. The 3 phase coil system can have further coil configurations so that they are balanced during the rotation of the trackable structure 130. Here, a coil group A1 may have another coil group A2 on the opposite side, and coil group B1 may have another coil group B2 on the opposite side, and coil group C1 may have another coil group C2 on the opposite side. For example, coil group A1 may overlap with coil group C2 and coil group B2, and coil group B1 may overlap with coil group A2 and coil group C2, and coil group C1 may overlap with coil group A2 and coil group B2, and so forth.

In further embodiments, the degree of overlap may be chosen with the consideration that coil group A does not couple to coil group B, and coil group B does not couple to coil group C, and coil group C does not couple to coil group A.

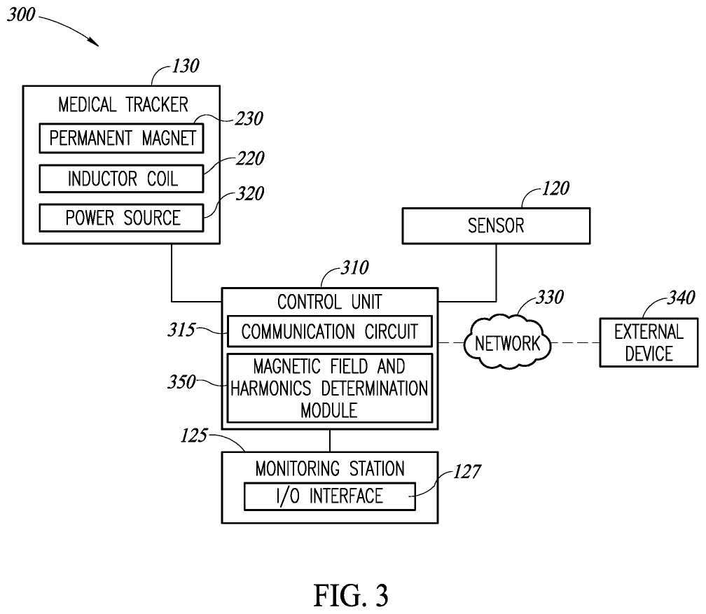

FIG. 3 is a block diagram of a medical tracking system 300 for detecting the position of a trackable structure 130 within a body of a patient, according to one or more embodiments. The system 300 includes one or more trackable structures (or medical trackers or medical tracking devices) 130, a sensor 120, a monitoring station 125, and a control unit 310. Other components may be included in the system 300. The control unit 310 is electrically connected to the trackable structure 130, the sensor 120, and the monitoring station 125. The trackable structure 130 includes at least one permanent magnet structure 230 and a power source 320. The at least one permanent magnet structure 230 is operatively coupled to at least one coil 220.

In some embodiments, one or more components of the system 300 are integrated. In other embodiments, two or more components of the system 300 are separate and distinct. For example, in at least one embodiment, the sensor 120, monitoring station 125, and control unit 310 are arranged in a single package (e.g., a single housing). In other embodiments, individual circuits of the components are separate and distinct while also cooperatively coupled. For example, in at least one embodiment, the control unit 310 includes one or more circuits integrated with the monitoring station 125 and one or more circuits integrated with the sensor 120.

In some embodiments, the at least one permanent magnet structure 230 includes one or more permanent magnets. Coils 220 are formed from one or more separate and distinct conductors.

In one embodiment, the trackable structure 130 is a medical device configured to be introduced, either partially or wholly, into the body of a patient in conjunction with a medical procedure. Representative but not exhaustive examples of medical instruments include complete, or portions of, cardiovascular devices and implants such as implantable cardioverter defibrillators, pacemakers, pacemaker leads, stents, stent grafts, bypass grafts, catheters and heart valves; orthopedic implants such as hip and knee prosthesis; spinal implants and hardware (spinal cages, screws, plates, pins, rods and artificial discs); a wide variety of medical tubes, cosmetic and/or aesthetic implants (e.g., breast implants, fillers); a wide variety of polymers, bone cements, bone fillers, scaffolds, and naturally occurring materials (e.g., heart valves, and grafts from other naturally occurring sources); intrauterine devices; orthopedic hardware (e.g., casts, braces, tensor bandages, external fixation devices, tensors, slings and supports) and internal hardware (e.g., K-wires, pins, screws, plates, and intramedullary devices (e.g., rods and nails)); cochlear implants; dental implants; medical polymers, a wide variety of neurological devices; fiducial markers; intravascular stylets (e.g., ECG stylets); stylets pre-loaded into respective catheters; central venous catheters; peripherally inserted central venous catheters; guidewires; thermal energy delivery devices; cryonic therapy delivery devices; photonic therapy delivery devices; cautery delivering catheters; balloon catheters; and other such devices. The trackable structure 130 can also include many other kinds of medical devices that can be introduced into the body of a patient as part of a medical procedure. The patient may be a human patient or a non-human patient (such as animals).

The monitoring station 125 includes an input/output device 127. In some embodiments, the monitoring station 125 is an input device only. In some cases, the monitoring station 125 is an output device only. For example, the input/output device may include in total or in part any one or more of a display, a keyboard, a mouse, a tactile apparatus (e.g., touchscreen, vibrator), a programmatic communication port (e.g., serial port such as a universal serial bus (USB) port, wireless transceiver such as a cellular-based radio, an IEEE 802.11 radio), an audio apparatus (e.g., microphone, speaker, piezo circuit device), or any other such input/output device. The monitoring station 125 may be contained in a single circuit or a plurality of distributed circuits, which may all be local, remote, or a combination of local and remote to each other. For example, in some embodiments, the monitoring station 125 includes a local display and a remote display communicatively coupled to the system 300 via a network such as the Internet.

In some embodiments, the control unit 310 generates a video signal and outputs the video signal to the monitoring station 125 (e.g., a display). The video signal includes a representation of the position of one or more trackable structures 130 within the body of the patient. The video signal can also include position data that can be displayed or otherwise presented via the monitoring station 125. The position data can include text that indicates numerical coordinates representing the amplitude (e.g., strength of the magnetic field), phase information, position, orientation, and motion of the trackable structure 130. The video signal displayed or otherwise presented via the monitoring station 125 can present in real time both a visual representation of the position of the trackable structure 130 within the body of the patient and certain position data that indicates the position of the trackable structure 130 within the body of the patient.

In some embodiments, in transmitting and receiving data for processing (e.g., sensor data received from sensors such as magnetometers), a communication circuit 315 included in the control unit 310 may communicate signals having information and data via a communication medium 330 (e.g., network, cloud, etc.) to and from an external device 340 (e.g., server, display device, etc.). The system 300 may communicate data (e.g., sensor data and position data processed from the sensor data, position of the trackable structure 130, XYZ components of the magnetic field, harmonics generated from the magnetic field, any other relevant images and signals, or the like) through any suitable communication scheme incorporated in the device or system. Various wireless communication schemes such as, for example, infrared, ZigBee, Ethernet, USB, Bluetooth, Wi-Fi, or near field communication (NFC), or cellular schemes such as CDMA, WiMAX, LTE may be used.

In some embodiments, the communication circuit 315 uses an antenna for sending and receiving information. The communication circuit 315 can optionally include one or more oscillators, encoders, decoders, amplifiers, filters, mixers, frequency injectors to modulate or demodulate information on a carrier frequency to be transmitted or received by the antenna. The antenna is operated by the control unit 310 to communicate information to and from the medical tracking device 100 and the external device 340.

In some embodiments, the trackable structure 130 is integrated with a medical instrument. For example, when the medical instrument includes or is a stylet, the trackable structure 130 may be formed as part of the stylet. In other embodiments, the trackable structure 130 is fixedly or removably coupled to the medical instrument. The range of cooperative combinations of medical instruments and trackable structure 130 is not limited merely to the combinations described herein, which are limited for brevity. Rather, the range of cooperative combinations of medical instruments and trackable structure 130 is broadly inclusive of those contemplated by one of ordinary skill in the art.

In many medical procedures, it is advantageous to track the position of the trackable structure 130 within the body of the patient with acceptable accuracy. For example, if the medical instrument is delivering fluid to a particular part of the patient's body, then it can be advantageous to accurately track the position of trackable structure 130 to ensure that the medical instrument is in the correct position for fluid delivery. In some particularly sensitive medical procedures, knowing the exact position of the trackable structure 130 with an acceptable level of accuracy can help ensure the well-being of the patient during a medical procedure.

The permanent magnet structure 230 enables tracking of the position of the trackable structure 130. When a current is passed through a coil 220 and the permanent magnet 230 rotates, a magnetic field is generated. The generated magnetic field which also generates harmonics can enable tracking of the trackable structure 130. In one or more embodiments, the medical tracking system may only need to generate one frequency of the magnetic field to provide viable tracking of the magnetic trackable structure.

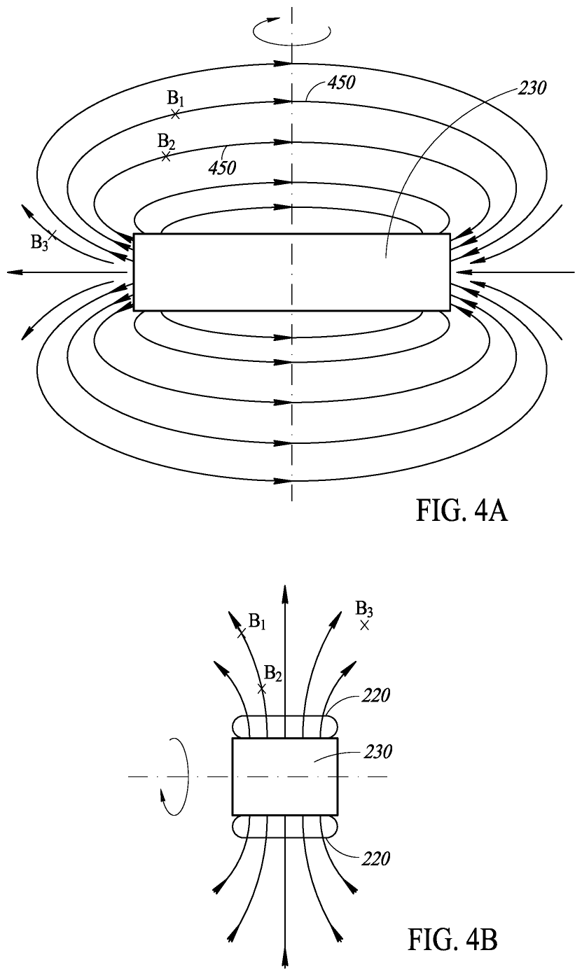

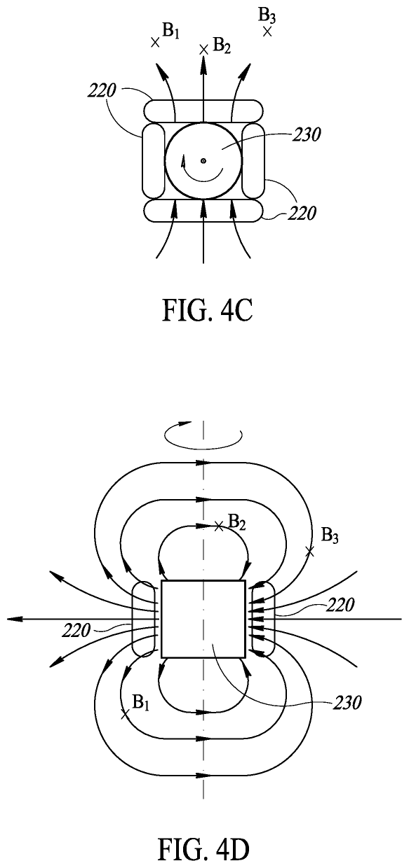

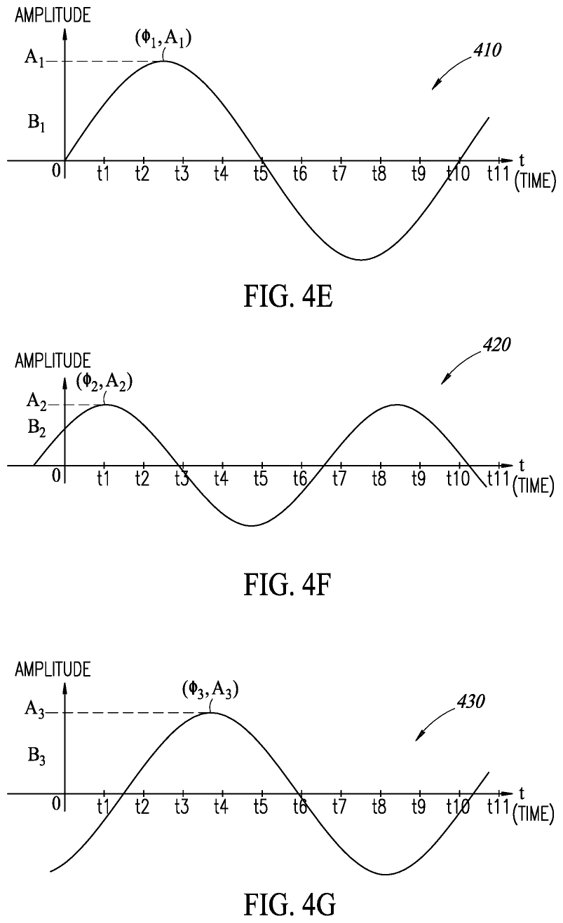

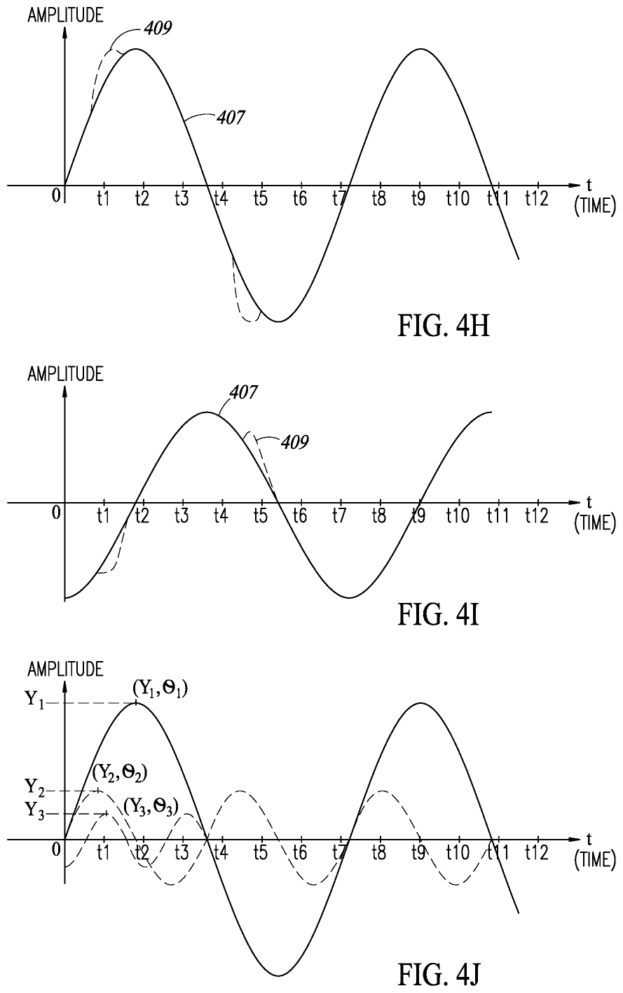

This will be further detailed in connection with FIGS. 4A to 4J.

The sensor 120 includes one or more magnetic sensors arranged to detect one or more magnetic fields generated by a coil 220 and/or the permanent magnet structure 230. The sensor 120 can detect certain parameters of the generated magnetic field such as field strength, polarity, orientation and direction of the magnetic flux as well as harmonics related to the generated magnetic field, and phase information of the generated magnetic fields. The sensor 120 generates one or more sensor signals indicative of the parameters of the aforementioned signals related to the generated magnetic field. The position of the trackable structure 130, and in some cases the position of two or more trackable structures 130, along with orientation, motion, and other location-based information can be determined based on the parameters of a magnetic field generated by the permanent magnet structure 230. Operations of the sensor 120 are in some cases coordinated by the control unit 310 such that parameters to direct certain sensor functions are applied in cooperation with parameters to direct excitation of the permanent magnet structure 230.

In one embodiment, the control unit 310 drives the coils 220 at a certain frequency and calculates location-based information (e.g., position, orientation, motion, timing, and the like) of a particular trackable structure 130. The control unit 310 receives one or more sensor signals from the sensor 120 and analyzes the one or more sensor signals. The control unit 310 generates the location-based information, such as the position, orientation of the trackable structure 130, based on the one or more sensor signals.

In one embodiment, control unit 310 executes particular algorithms to identify and track the position of trackable structures 130 in three dimensions and the orientation of trackable structures 130 relative to a reference point. For example, the 6 DOF location of the trackable structure 130 may be obtained with respect to a reference coil or to a drive coil. The control unit 310 may register the alignments of the trackable structure 130 via a memory storage (not shown). In some embodiments, the rotation information including the rotational axis of the trackable structure 130 are obtained and provided to the control unit 310 to determine the phase relationship associated with the trackable structure 130. The memory storage may be a non-transitory computer-readable medium that can include, without limitation, magnetic disks, optical disks, organic memory, or any other volatile (e.g., RAM) or non-volatile (e.g., ROM) storage system readable by the processor within the control unit 310. The memory can include a data storage to store indications of data, such as sensor readings (e.g., related to the magnetometer), parameters, algorithms, executable program instructions, and other settings, for example.

The identification and tracking of one or more trackable structures 130 by a control unit 310 is based, at least in part, on the position of the permanent magnet structure 230. In these and other embodiments, tracking the position of a trackable structure 130 includes integrating the frequency, amplitude, and phase information of the magnetic field sensed from sensors at various locations at a certain point in time.

It can be difficult to accurately track the position of a conventional trackable medical device within the body of a patient if the trackable medical device is positioned deep within the body of the patient. In larger patients, for example, the problem can be exacerbated because the trackable medical device may need to travel deeper below the skin and deeply into the body of the patient in order to reach particular areas inside the body in accordance with various medical procedures. It can be difficult to generate a magnetic field with sufficient strength and stability to allow reliable tracking of the trackable medical device. This problem in the conventional technology can be compounded by the fact that in many circumstances it is more desirable to have a coil and a core that are relatively small in order to reduce or minimize disruption of body tissues as the trackable medical device is introduced into the body of the patient. This problem can also be compounded by naturally occurring magnetic fields (e.g., the earth's magnetic field) and man-made magnetically disruptive structures such as bed frames and other ferrous medical devices. As the dimensions of the coil are reduced, it can be difficult to generate sufficiently strong and acceptably stable magnetic fields to enable detection. Furthermore, interference as described herein (e.g., from the earth's magnetic field, from other medical and non-medical equipment positioned in or near the patient's body), and even interference from the trackable medical device itself can make it difficult to calculate the position of the trackable medical device within the body of the patient with acceptable accuracy. The rotating permanent magnet structure 230 according to the present disclosure resolves the technical problem faced in the conventional art.

In one or more embodiments, in order to accurately track the trackable structure 130 deep within the body of a patient, the control unit 310 drives the coil 220 with a low-frequency alternating current (AC) excitation signal instead of a direct current (DC) signal or a high-frequency excitation signal. The low-frequency excitation signal causes a current to be passed through the coil 220 and the permanent magnet 230 rotates. The magnetic field generated by the permanent magnet structure 230 has particular characteristics based in part on a waveform of the excitation signal. These particular harmonics, oscillating characteristics can enable the control unit 310 to distinguish the generated magnetic field from noise, interference, and/or magnetic fields produced by devices or circumstances different from the permanent magnet structure 230. In this way, the control unit 310 can track the position of the trackable structure 130 with acceptable accuracy even when the trackable structure 130 is deep within the body of a patient.

In at least one embodiment, the control unit 310 drives the coil 220 with an excitation signal having a frequency less than about 2,500 Hz. In some embodiments, the control unit 310 drives the coil 220 with an excitation signal having a frequency of between about 200 Hz and 700 Hz. The selection of a 330 Hz excitation signal, for example, helps to avoid AC line related components, which might occur at a multiple of a line frequency. For example, 300 Hz, which is a multiple of both 50 Hz and 60 Hz, which are two common line frequencies conventionally used in Europe and the U.S., respectively, may provide strong magnetic returns, but the strong magnetic returns may also have measurable harmonic components associated with the AC line frequency. For at least these reasons, some embodiments select an excitation signal having a frequency below 700 Hz, near 330 Hz, and in avoidance of integer multiples of a common line frequency.

Control unit 310 has been described as driving a coil 220 with an excitation signal or applying an excitation signal to a coil 220. The control unit 310 can accomplish this by directly applying the excitation signal to the coil 220. Alternatively, the control unit 310 can accomplish this indirectly by controlling a voltage source that applies a voltage to the coil 220 or by controlling a current source that supplies a current to the coil 220. Those of skill in the art will recognize, in light of the present disclosure, that the control unit 310 can generate, pass, or otherwise apply an excitation signal to the coil 220 in many other ways. All such other ways are within the scope of the present disclosure.

In at least one embodiment, the monitoring station 125 includes a display that presents a visual representation of the position of one or more trackable structures 130 within the body of the patient. The visual representation of the position of a trackable structure 130 enables a medical practitioner to know the position of the trackable structure 130 within the body of the patient with acceptable accuracy. This in turn can enable the medical practitioner to correctly perform medical procedures on the patient.

The control unit 310 may include multiple discrete control circuit portions. Control unit 310 can include one or more microcontrollers, one or more microprocessors, one or more memory devices, one or more voltage sources, one or more current sources, one or more analog-to-digital converters, one or more digital-to-analog converters, and/or one or more wireless transceivers. One or more of these components can collectively make up the control unit 310.

In addition, the processor included in the control unit 310 may be any hardware device or any computer processor operable to execute instructions (e.g., stored in memory) and/or processing data to perform the functions of the medical tracking device 100 as described herein. For example, the processor may include microcontrollers, microprocessors, reduced instruction set computers (RISC), application specific integrated circuits (ASICs), field-programmable gate arrays (FPGAs), graphical processing units (GPUs), logic circuits, or other similar devices capable of executing the functions described herein.

The control unit 310 further includes a magnetic field and harmonics determination module 350. The magnetic field and harmonics determination module 350 may be implemented with one or more of the microprocessors/processors discussed above. The details and the operations of the magnetic field and harmonics determination module 350 will be explained in connection with FIGS. 4A to 4J.

The power source 320 provides power for the medical tracker 130 to provide the rotational force to the permanent magnet 230. In some embodiments, the power source 320 includes a rechargeable battery such as a flexible, thin-film, or solid-state battery (e.g., Li-ion battery).

In other embodiments, the power source 320 may be arranged outside of the medical tracker 130 and provide power for the control unit 310, the sensor 120, the monitoring station 125, and other components such as display, memory, and other components within the system 300 or the medical tracking device 100.

The medical tracking system 300 may further include mechanisms or modules for monitoring the phase of the spinning magnet 230. This may be included as part of the medical tracker 130, the sensor 120, or the control unit 310. In other embodiments, it may be communicatively or functionally coupled to one of the medical tracker 130, the sensor 120, or the control unit 310 to monitor the phase of the spinning magnet. For example, inductive pickup, optical transmitter/receiver, or other suitable devices may be used to monitor the phase of the magnet 230. This phase information may then be provided to the control unit 310 for processing.

In further embodiments, a reactive magnetic element 950 may be positioned adjacent to the medical tracker 130. These reactive magnetic asymmetry near the spinning magnet 230 are used to generate magnetic harmonics that indicate phase.

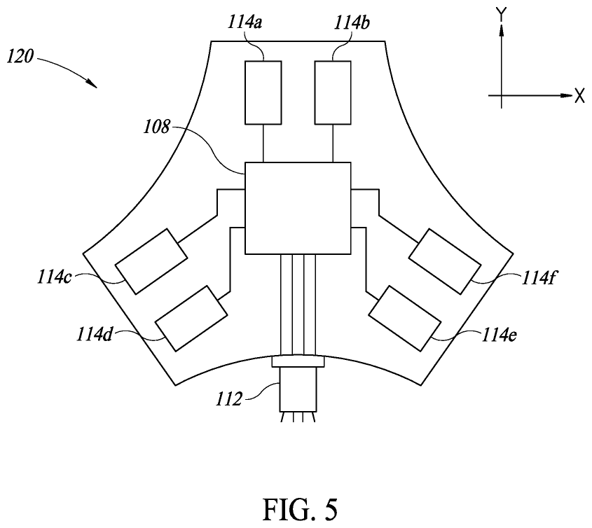

The details of the sensor 120 will be explained in connection with FIG. 5. FIG. 4A is a cross-sectional view of a trackable structure 130 and the magnetic field lines 450, according to one or more embodiments. FIGS. 4B, 4C, 4D are cross-sectional views of various embodiments of a trackable structure 130 showing the relationship between the magnetic field lines 450 generated from a magnetic structure 230 with respect to the axis of spin and the arrangements of the coils 220. FIGS. 4E, 4F, 4G are graphs of magnetic fields of a trackable structure 130 collected from sensors at various locations, according to one or more embodiments.

FIG. 4A illustrates the trackable structure 130 having a permanent magnet 230 and coils 220 arranged adjacent to a rotating permanent magnet 230. One example of the magnetic lines 450 of the magnetic fields formed based on the rotating permanent magnet 230 is shown. The strength of the magnetic field illustrated in FIG. 4A at any given location is representatively illustrated by the density of the magnetic field lines 450. In particular, where magnetic field lines 450 are closer together, the magnetic field is stronger. Where magnetic field lines 450 are further apart from each other, the magnetic field is weaker. The direction of the magnetic field is indicated by the direction of the arrows on the magnetic field lines 450 at any given location. A person of ordinary skill in the art will readily appreciate that there are infinite number of magnetic flux lines 450 present around the magnet.

In one or more embodiments, the magnetic field lines 450 coming from the core of the magnet 230 penetrates through a drive coil surface of the drive coils 220. As shown in FIG. 4A, the axis of spin that is the vertical line with the arrow that indicates the direction of the spin for the core of the magnet 230 may be transverse to the direction of magnetic field lines 450 coming from the magnet's poles. The ratio of LID in FIG. 4A is about 0.25. Namely, the rotating magnetic has a diameter D that is about 4 times larger than its length L. Further embodiments of the relationship of the magnetic field lines 450 generated from the magnet 230 with respect to the axis of spin and the arrangements of the coils 220 are illustrated in FIGS. 4B, 4C, and 4D. FIGS. 4B and 4D are cross-sections made parallel from the axis of spin. FIG. 4C is a cross-section made perpendicular from the axis of spin. In FIGS. 4B and 4D, the ratio between the longitudinal length L and the diameter D of the magnetic structure 230 (L/D) as illustrated is close to 1. However, the L/D ratio of the magnetic structure 230 may have wider L/D ratio range. For example, the L/D ratio of the magnetic structure may vary from 0.5<<5. Other L/D ratio out of this range may also be contemplated without deviating from the spirit and scope of the present disclosure. In one or more embodiments, the sensor 120 (see FIG. 1) may have one or more magnetometer or magnet sensors to sense the magnetic fields (including harmonics of the magnetic fields) at different locations adjacent to the trackable structure 130. For example, magnetometer B₁, magnetometer B₂, magnetometer B₃ may be positioned as shown in FIG. 4A to detect the magnetic field at that particular location. In one or more embodiments, the magnetometer B₁, magnetometer B₂, magnetometer B₃ may be one of a plurality of magnetic sensors 114a-114f within the sensor 120 (see FIG. 5). The particular location in which the magnetometer B₁, magnetometer B₂, magnetometer B₃ are drawn to be placed within each FIGS. 4A, 4B, 4C, and 4D may be physically different locations from each other. For example, the location of B₁ in FIG. 4A may be different from the location of B₁ in FIG. 4B.

Graph 410 in FIG. 4E, Graph 420 in FIG. 4F, and Graph 430 in FIG. 4G shows the magnetic field generated by the rotating permanent magnet 230 at a point in time. Harmonics may be included in these generated magnetic fields due to certain imperfections and designed-in choices. For example, the ferromagnetic materials in the clinical setting may cause harmonics as well as noise, interference, the earth's magnetic field, electromagnetic interference from other medical and non-medical equipment that may be positioned in or near the patient's body, from electronic circuitry, and from the medical instrument or the trackable structure 130 itself, and/or magnetic fields produced by other devices different from the permanent magnet structure 230 may cause harmonics. However, these harmonics are not shown in Graph 410 in FIG. 4E, Graph 420 in FIG. 4F, and Graph 430 in FIG. 4G for simplicity reasons and will be further shown and explained in connection with FIGS. 4H to 4J.

For example, Graph 410 of FIG. 4E shows a sinusoidal magnetic field at a location where the magnetometer B1 is arranged. The magnetic field at this location has a magnitude/amplitude information A₁, phase information ϕ₁. Graph 420 of FIG. 4F shows a sinusoidal magnetic field at a location where the magnetometer B₂ is arranged. The magnetic field at this location has an amplitude information A₂, phase information ϕ₂. Graph 430 of FIG. 4G shows a sinusoidal magnetic field at a location where the magnetometer B₃ is arranged. The magnetic field at this location has an amplitude information A₃, phase information ϕ₃. The amplitudes of A₁, A₂, and A₃ may be different from each other and the phase information ϕ₁, ϕ₂, and ϕ₃ may be different from each other based on the different locations of the magnetometers B₁, B₂, B₃. In some embodiments, the magnetometers B₁, B₂, B₃ can be employed to get the magnitude, phase, and the orientation information from an x-axis perspective Bx, a y-axis perspective By, and a z-axis perspective Bz, respectively.

The magnetic field is stronger where magnetic field lines 450 are closer together, accordingly the amplitude readings A₃ of the magnetometer B₃ may be higher than the amplitude A₁ of the magnetometer B₁ and the amplitude A₂ of the magnetometer B₂. Further, because the magnetic field is weaker where magnetic field lines 450 are further apart from each other, the amplitude readings A₁ of the magnetometer B₁ may be smaller than the amplitude A₂ of the magnetometer B₂.

In some embodiments, the frequency of the detected sinusoidal magnetic fields from each of the magnetometers B₁, B₂, B₃ all have substantially the same frequency. In some embodiments, the frequency of the detected sinusoidal magnetic fields from each of the magnetometers B₁, B₂, B₃ are the same as the trackable structure 130 that receives excitation signals from the coils 220 that have a certain set frequency. Accordingly, while the trackable structure 130 may be operated at different frequencies, the frequencies of the magnetic fields may nevertheless have substantially the same frequency but with different phase and amplitude information. Further, in one or more embodiments, the magnetometers B₁, B₂, B₃ are capable of detecting the orientation of the magnetic field (for example, in the form of a vector) at the location where the magnetometers are placed.

The sensor 120 or the magnetometers B₁, B₂, B₃ included in the sensor 120 detects the frequency information, orientation information, amplitude information, and phase information from more than one point in time (for example, a first snap shot at time t1 and a second snap shot at time t2) and the magnetic field and harmonics determination module 350 of the control unit 310 may calculate the location of the spinning or rotating permanent magnet 230. By having the frequency, orientation, amplitude, and phase information at time t1 and at time t2, and comparing this information, it is possible to determine the entire magnetic field caused by the magnetic dipole of the rotating permanent magnet 230. By gaining the entire magnetic field information created by the dipole, the magnetic field and harmonics determination module 350 may in turn determine the accurate location of the dipole which will be where the rotating permanent magnet 230 will be at. Further, in this method, the effect of DC fields or any magnetic interferences caused by ferromagnetic materials in the clinical setting can be disregarded. For example, subtracting the information obtained at time t2 with time t1, the magnetic field and harmonics determination module 350 may remove the impact of any DC field and sense the AC magnetic field of the rotating magnet.

In further embodiments, more than one trackable structure 130 can be used. For example, a first trackable structure may be spinning at 330 Hz and a second trackable structure may be spinning at 420 Hz with the same patient at the same time. Accordingly, if a patient has two trackable structures inside the patient's body, the first and second trackable structures operating at different frequencies, it will be possible for the sensor 120 to detect the locations of the first and second trackable structures. Additional trackable structures 130 may be inserted into the patient's body as needed and the accurate locations of these trackable structures 130 all operating at a different frequency from each other may be detected.

In some embodiments, the medical tracking system 300 is capable of a 5 DOF tracking. That is, in some cases where the harmonics are not generated, the medical tracking system 300 is still capable of providing 5 DOF information regarding the trackable structure 130 using the fundamental rotating frequencies of the rotating trackable structure 130 (or to be exact the rotating permanent magnet 230 included within the trackable structure 130) as described above. However, if rotor phase information (phase information of the tracked magnetic structure), is obtained, then the medical tracking system 300 can perform 6 DOF tracking (see FIGS. 4H to 4J and its relevant descriptions). Further, the medical tracking system 300 according to the present disclosure may obtain 6 DOF information of the trackable structure 130, the harmonics associated with the magnetic field need not be used to locate the position of the rotating magnetic structure 130. That is, in some embodiments, even without using the harmonics, obtaining the 6 DOF information is possible based on the fundamental frequency of the magnetic field and the phase information of the tracked magnetic structure and the sensor tracking the structure.

In one or more embodiments, the medical tracking system 300 need only generate one frequency of the magnetic field to provide viable tracking of the rotating magnetic structure 130.

FIG. 4H is a graph showing harmonics included in the magnetic fields generated by a trackable structure 130 as detected by a sensor 120 at a first location, according to one or more embodiments. FIG. 4I is a graph showing harmonics included in the magnetic fields generated by a trackable structure 130 as detected by a sensor 120 at a second location, according to one or more embodiments. FIG. 4J is a graph showing various harmonics of various frequencies with different phase and amplitude information.

FIGS. 4H, 4I illustrate a sinusoidal magnetic field 407 (shown as bolded lines) showing harmonics 409 (shown as dotted lines) detected from one of the magnetic sensors B₁, B₂, B₃ at one location. FIG. 4J illustrates a sinusoidal magnetic field (shown as bolded lines) showing harmonics (shown as dotted lines) detected from one of the magnetic sensors B₁, B₂, B₃ at a different location. FIG. 4J illustrates a more accurate and appropriate representation of the generated magnetic fields and the harmonics sensed from the magnetic sensors B₁, B₂, B₃.

Non-sinusoidal complex waveforms such as those shown in FIGS. 4H and 41 are constructed by addition or superposition of a series of sine wave frequencies known as harmonics (see FIG. 4J). Harmonics are used to describe the distortion of a sinusoidal waveform by waveforms of different frequencies. This complex, distorted sinusoidal waveform can be split up mathematically into its individual components called a fundamental frequency and a number of harmonic frequencies.

A fundamental waveform (or first harmonic) is the sinusoidal waveform that has the fundamental frequency f (the lowest or base frequency) on which the complex, distorted waveform (e.g., non-sinusoidal wave pattern) is built and as such the periodic time, T of the resulting complex waveform will be equal to the periodic time of the fundamental frequency. Referring to FIG. 4J, a sinusoidal waveform (shown in bolded line) is an alternating signal, which varies as a sine function of angle, 2πf. The waveforms frequency, f is determined by the number of cycles per second. Harmonics, on the other hand, are signals that operate at a frequency that is an integer (whole-number) multiple of the fundamental frequency. For example, given a 60 Hz fundamental waveform, this means a second harmonic frequency would be 120 Hz (2×60 Hz), a third harmonic would be 180 Hz (3×60 Hz), and so on. Namely, the harmonics are multiples of the fundamental frequency and can therefore be expressed as: 2f, 3f, 4f, etc.

In FIG. 4J, the fundamental waveform having frequency f has an amplitude of Y₁ and a phase of θ₁. The second harmonic has a 2f frequency, an amplitude of Y₂ and a phase of θ₂. Other harmonics with a multiple of the fundamental frequency with different amplitude and phase information may exist although not shown in FIG. 4J for brevity.

These harmonics shown in FIG. 4J, which make up for the distorted wave patterns shown in FIGS. 4H and 4I, are used to track the location of the trackable structure 130. That is, while the various noises and interferences associated with the clinical circumstances (e.g., magnetic materials affecting the permanent magnet during a clinical surgery, significant vibrations caused by the rotation of the permanent magnet, vibrations or oscillations of the adjacent medical devices, or the like) causes the harmonics, these harmonics are beneficial in detecting the location of the trackable structure 130.

One or more methods of tracking based on rotating hard magnetic materials that create harmonics in the magnetic field is described as follows. A person of ordinary skill in the art would readily understand that there may be various ways of accomplishing the tracking of the rotating magnetic materials based on the present disclosure.

One method of detecting the 6 DOF from the harmonics is to utilize the Goertzel algorithm or windowed FFT (fast fourier transform).

The Goertzel algorithm is one of many techniques in digital signal processing (DSP) for efficient evaluation of the individual terms of the discrete Fourier transform (DFT). For example, the Goertzel algorithm may be able to analyze one selectable frequency component from a discrete signal. Further, the Goertzel algorithm applies a single real-valued coefficient at each iteration, using real-valued arithmetic for real-valued input sequences. For covering a full spectrum, the Goertzel algorithm has a higher order of complexity than fast Fourier transform (FFT) algorithms, but for computing a small number of selected frequency components, it is more numerically efficient. In one or more embodiments, the simple structure of the Goertzel algorithm makes it well suited to small processors and embedded applications.

Based on the magnetic sensors, it is possible to acquire the magnitude and frequency of the signals generated from the magnetic field. If the magnitude and frequency are determined, then the phase is determinable with only two sample times. It does not matter that time is quantized so long as it is known what that quantization is and the sample times are chosen to not line up with certain special conditions (such as both readings being zero). By obtaining the magnitude and frequency information at a first sensing time (or a first sampling time) and at a second sensing time (or a second sampling time), by applying the Goertzel algorithm or windowed FFT, the phase information may also be obtained. With the magnitude, frequency, and phase information, determining the magnetic field generated by the magnetic dipole (or the spinning permanent magnet) is available.

In some embodiments, sampling of the sensed information (e.g., magnitude, frequency, orientation, and phase information) may only happen at an appropriate time. For example, the sampling may happen at 4 samples per cycle (±, 0,90°). That is, one point may happen when the signal phase is at a +phase, a second point may happen when the signal phase is at a −phase, a third point may happen when the signal phase is at 0, and a fourth point may happen when the signal phase is at 90°. In this case, every magnetic sensor may produce at least 2 magnetic field readings and may be used to obtain data on the 6 DOF spinning object. Thus, samples can be taken at any desired time, such as t1, t2, t3, t4, etc. and the location of the sensor known exactly.

In some embodiments, analyzing the harmonics to obtain at least one of an amplitude information, orientation information, phase information, and frequency information generated based on the magnetic field may be conducted multiple times. The magnetic dipole (e.g., a low frequency trackable structure 130) may be located based on the difference of the at least one of the amplitude information, orientation information, phase information, and frequency information at multiple times. The microprocessor (included in the control unit 310 or included in the control circuit 108) then determines the location of the magnetic dipole as the location of the low-frequency trackable structure 130.

Yet in some embodiments for determining the 6 DOF of the spinning permanent magnet, amplitude and phase information may be used. In particular, the amplitude and phase information is utilized to generate 2 channels of data per frequency channel. By obtaining this data and applying either the windowed FFT or Goertzel algorithm, obtaining the positional information of the permanent magnet is possible.

In some embodiments, an asymmetry could be exploited to provide 6 DOF. For example, the drive coils are one such asymmetry. Adding a ferromagnetic material at some rotation could provide a second asymmetry. In some cases, the phase of the rotor may not necessarily be aligned with the phase of the drive (e.g., lag leads to the torque that compensates for frictional losses and dynamic behaviors). The protective conductive coating that is typically on neodymium magnets may likely lead to a damping of oscillations about the nominal rotation frequency (e.g., better stability). In some cases, pulse width modulation (PWM) of the drive coils may allow for smoothing out the applied torques on the rotor. The use of PWM pulses to drive a rotor are well-known and thus need not be described here.

However, in other embodiments, symmetry may be utilized in determining the 6 DOF of the permanent magnet.

FIG. 5 illustrates a sensor 120, according to one or more embodiments. The sensor 120 is arranged in a housing with a plurality of magnetic sensors 114a-114f and a control circuit 108, according to at least one embodiment. The sensor 120 includes sensor portions comprising a plurality of magnetic sensors 114a-114f, a control circuit 108, and an electrical conduit 112. The control circuit 108 is coupled to the sensor portions 114a-114f and to the electrical conduit 112.

In at least one embodiment, the control circuit 108 outputs an excitation signal to a coil 220 of a trackable structure 130. The excitation signal causes the permanent magnet structure 230 to rotate and thereby generate a magnetic field as described in the present disclosure.

In the embodiment of FIG. 5, the sensor 120 includes six sensor portions, each sensor portion having at least one magnetic sensor (or magnetometer) 114a-114f. The six sensor portions are configured to collectively sense parameters (including amplitude, phase, orientation, frequency, etc.) of the generated magnetic field and other magnetic energy as well as harmonics associated with the generated magnetic field. The six sensor portions also generate sensor signals relative to the parameters of the generated magnetic field mentioned above. Each of the sensor portions passes at least one sensor signal to the control circuit 108. The control circuit 108 analyzes the sensor signals and calculates position information associated with the trackable structure 130 based on the sensor signals. The calculated position information may include information regarding the parameters mentioned above as well as a position of the trackable structure 130 in three-dimensional space, a position of the medical instrument represented in two-dimensional space, an orientation of the trackable structure 130, motion of the trackable structure 130, and other position information. The sensor signals are indicative of the parameters of the magnetic field generated by the permanent magnet structure 230.

In some embodiments, in addition to the computing resources provided in the control circuit 108, additional and different computing resources are employed. For example, the control circuit 108 may provide preliminary collection, aggregation, or other processing of sensor data, and the control circuit 108 may communicate certain data (e.g., some or all of the collected, aggregated, and processed sensor data) to a remote computing device (not shown) such as a laptop computer, a cloud computing device, an ultrasound or other imaging medical equipment, or some other computing device. The remote computing device may provide additional processing to generate position data, video data, audio data, tactile data, image or other signal processing, and the like. In one or more embodiments, the control circuit 108 may be implemented in the same manner as the control unit 310. For example, the control circuit 108 may include microcontrollers, microprocessors, reduced instruction set computers (RISC), application specific integrated circuits (ASICs), field-programmable gate arrays (FPGAs), graphical processing units (GPUs), logic circuits, or other similar devices capable of executing the functions described above.

While FIG. 5 shows six sensor portions, the sensor 120 can include more or fewer individual sensor portions than shown in FIG. 5. Accordingly, the sensor 120 may also include more or fewer magnetic sensors 114a-114f as shown in FIG. 5. For example, there may be 10 sensors sensing the magnetic field along an x-axis and 10 sensors sensing the magnetic field along a y-axis and another 10 sensors sensing the magnetic field along a z-axis. However, as mentioned, less than 30 sensors may be used in different settings and applications.

In at least one embodiment, the magnetic sensors 114a-114f are configured to sense the magnitude of the generated magnetic field in all three spatial dimensions. For example, magnetic sensors 114a, 114b are configured to sense a magnitude of certain components the generated magnetic field along a first axis (e.g., x-axis). Magnetic sensors 114c, 114d are configured to sense the magnitude of certain components of the generated magnetic field along a second axis (e.g., y-axis) orthogonal to the first axis. And magnetic sensors 114e, 114f are configured to sense a magnitude of certain components of the generated magnetic field along a third axis (e.g., z-axis) orthogonal to the first axis and the second axis.

In at least one embodiment, each pair of sensor portions provide a differential sensor signal in order to enhance accurate detection of the generated magnetic field along each of the three axes. For example, magnetic sensors 114a, 114b output sensor signals having opposite polarities. Magnetic sensors 114c, 114d output sensor signals having opposite polarities. And magnetic sensors 114e, 114f output sensor signals having opposite polarities. In this way, the six sensor portions enable accurate detection of parameters of the generated magnetic field in three spatial dimensions.

In at least one embodiment, the magnetic sensors 114a-114f include magnetoresistive sensors. The magnetoresistive sensors include materials whose electrical resistance varies in accordance with a magnetic field proximal to and sensed by the magnetoresistive sensors. In some embodiments, for example, magnetic sensors 114a-114f provide sufficiently accurate sense data to the control circuit 108 to detect and track with acceptable accuracy a trackable structure 130 that is in the body of a patient 140 when the sensor 120 is within 25 centimeters (cm) of the trackable structure 130. In some embodiments, magnetic sensors 114a-114f provide sufficiently accurate sense data to the control circuit 108 to detect and track with acceptable accuracy a trackable structure 130 that is in the body of a patient 140 when the sensor 120 is within 10 cm, 25 cm, 30 cm, 50 cm, 65 cm or some other even greater distance of the trackable structure 130.

Each of the sensor portions can be configured to be sensitive to magnetic fields along a particular axis. The magnetic sensors 114a, 114b can be sensitive to magnetic fields along a first axis. The magnetic sensors 114c, 114d can be sensitive to magnetic fields along a second axis orthogonal to the first axis. The magnetic sensors 114e, 114f can be sensitive to magnetic fields along a third axis orthogonal to the first axis and the second axis. Thus, the three pairs of sensor portions are each configured to produce an electrical resistance that changes based on the strength of the targeted magnetic field in a particular direction.

In one embodiment, the magnetic sensors 114a-114f can include giant magnetoresistive (GMR) sensors. Additionally or alternatively, the magnetic sensors 114a-114f can include anisotropic magnetoresistance sensors, some other type of magnetoresistance sensors, or magnetic sensors based on another type of magnetic field measurement architecture.

In one or more embodiment, the sensor signal detected from the plurality of first magnetometers 114a, 114b, the plurality of second magnetometers 114c, 114d, and the plurality of third magnetometers 114e, 114f includes at least one of an amplitude information, orientation information, phase information, and frequency information generated based on the magnetic field. The sensor signal includes a first sensor signal at one relative phase and a second sensor signal detected at a second relative phase from the first. For instance, for a given frequency, an amplitude+phase can be generated for each sensor signal, the phase can be considered relative to a synchronization signal, and with a chosen additional phase delay, the control unit 310 or the control circuit 108 can determine for two orientations indicative of the rotational direction.

The arrangements of the magnetic sensors in the embodiments described above have been arranged that the group of magnetic sensors are aligned to have directions that are orthogonal to each other. However, a person of ordinary skill in the art would readily appreciate that the arrangement of the magnetic sensors does not have to be all orthogonal to each other, and it may have various arrangements. For example, the medical tracking system would operate with sensors 120 that include a plurality of magnetic sensors that are arranged in only one direction or two directions. Further, a plurality of magnetic sensors arranged in more than three directions may also be utilized. That is, the magnetic sensors may arranged in a large number of directions that are not orthogonal to each other within the sensor 120.

In at least one embodiment, the sensor portions of FIG. 5 include one or more coils. The coils can be positioned and oriented to be sensitive to magnetic field components in respective spatial directions. The signal to noise ratio (SNR) scales according to Formula (1),

(n^{(1/2)}*d^{(3/2)})*(f*Bo)*(BW^{(-1/2)}) (1)

wherein the particular parameters (e.g., size, diameter) of wire used to make the sensing coils is fixed. In Formula (1), the first two parameters are linked to the geometry of the sensing coil: “n” is the number of turns, “d” is the diameter of the coil. Also in Formula (1), “f” is the frequency of oscillation, “Bo” is the field that a magnet structure can support. Further still in Formula (1), BW is the filtered bandwidth, for example, an update rate of 3-30 Hz.

Still considering Formula (1), if the sensing coil is grown to 1 cm diameter then a factor of 2× improvement in SNR (200 turns) (+26% range) may be realized. Increasing the wire gauge or number of turns could further improve SNR (albeit slowly, 10× more turns=+47% range) assuming that environmental noise and coil resonance does not limit the sensitivity. In one embodiment, the circuitry in the sensor 120 can allow detection and generation of position information of the trackable structure 130 up to 65 cm away from the sensor 120 with acceptable accuracy.

An additional consideration in the sensor 120 that includes the sensor portions and the control circuit 108 is the limited space available within the housing of the sensor 120. The limited space within the housing may dictate that relatively small coils be used in magnetic sensors 114a-114f If the size of the sensor housing is increased, such as if very large coils are used, for example, then there can be complications due to parasitic capacitance of the coils. This can possibly result in a natural resonant frequency and the potential to generate excessively large signals to later be filtered off. In at least one embodiment, the sensor portions can include a magnetic gain medium (e.g., ferromagnetic cores within the sensor coils). A magnetic gain medium can help to improve the SNR of the magnetic sensors 114a-114f. Furthermore, the size of gain media can be a limiting factor. For example, increasing the size of the sensor coils and the gain media may lead to increasing interference between adjacent sensing coils. In this way, the sensor 120 may provide a balanced geometry, which is a geometry that considers sizes, materials, distances between, orientations, and other such parameters of components of the sensor portions.

In at least one embodiment, the sensor portions include permanent magnets.

The sensor portions of FIG. 5 that include magnetic sensors 114a-114f are arranged to pass sensor signals to the control circuit 108. The control circuit 108 receives the sensor signals and analyzes the sensor signals. The control circuit 108 determines location-based information associated with the trackable structure 130 within the body of the patient 140 based on the sensor signals. The control circuit 108 can output any or all of a video signal, an audio signal, a tactile signal, or any other user or machine-perceptible signal indicative of or otherwise representing some or all of the location-based information. The location-based information may represent position data indicating the position of the trackable structure 130 within the body of the patient 140, an orientation of the trackable structure 130 relative to one or more reference points (e.g., a structure in or about the patient's body, a point on the sensor housing, and the like), actual or relative movement of the trackable structure 130, a historical track of previous positions of the trackable structure 130, a predicted track of the future position of the trackable structure 130, a velocity or other rate of motion information, and other like information associated with the trackable structure 130. In some cases, at least some of the location-based information is represented by a time-varying signal such as an audio signal of varying frequency to represent speed, distance, proximity to another structure, or the like. In some cases, at least some of the location-based information is represented by color or grayscale (e.g., lighter colors representing further distance and bolder colors representing closer distance). Many other representations are also contemplated.

In one embodiment, the sensor 120 can output data signals, control signals, excitation signals, power signals, or other types of signals via the electrical conduit 112. In particular, the sensor 120 can output signals to the trackable structure 130 and/or to a monitoring station 125. The sensor 120 can also receive signals from the permanent magnet structure 230, the monitoring station 125, and/or additional electronic equipment via the electrical conduit 112.

In at least one embodiment, the sensor 120 includes one or more wireless transmitters and/or receivers (not shown). Sensor 120 can transmit, receive, or transmit and receive wireless signals from one or more wireless transmitters and/or receivers. In particular, wireless transmitters and receivers (e.g., transceivers) can transmit and receive signals to and from the permanent magnet structure 230 and the monitoring station 125.

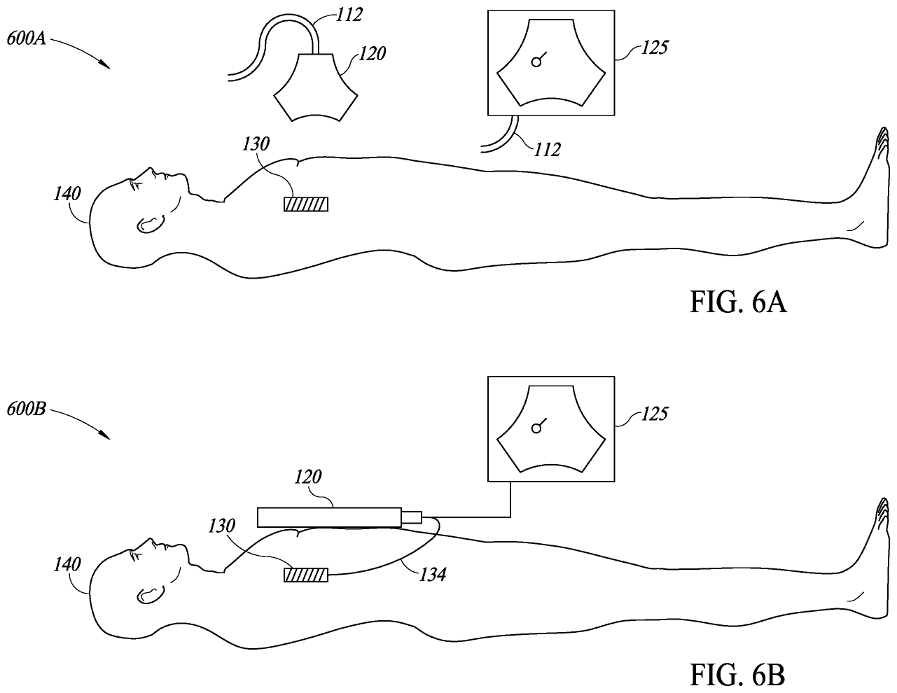

FIG. 6A illustrates a low-frequency medical tracking system 600A for detecting a position of a trackable structure 130 within a body of a patient 140, according to at least one embodiment. The low-frequency tracking system 600A may include components substantially along the lines of the system 300 for detecting the position of a trackable structure 130 within the body of a patient 140 in FIG. 3. A patient 140 is undergoing a medical procedure. The patient 140 may be a human patient or a non-human patient. A medical practitioner (not shown) is administering the procedure. The medical practitioner has placed a trackable structure 130, which may be embodied as a medical instrument, into the body of the patient 140. The trackable structure 130 may be implemented to be included in a stylet, a catheter such as a Peripherally Inserted Central Catheter (PICC), a medical tube, a tracheal tube, a needle, a cannula, or some other structure. For example, the trackable structure 130 may be implemented as a capsule type that has no wires connected to and from the trackable structure 130. In this case, the trackable structure 130 may include a microprocessor, a communication chip capable of also communicating data to and from the sensor 120 or the control unit 310 or the monitoring station 125.

In one or more embodiments, the trackable structure 130 implemented as a capsule stylet may be powered on or powered off to start or stop the rotation of the permanent magnet 230. For example, the wireless or cordless capsule stylet may be controlled to be powered ON/OFF by utilizing light with the use of a photodiode. Further example for controlling the power of the capsule stylet includes the user of a mechanical trigger, mechanical switch, magnetic trigger, radiation (e.g., the use of X-ray, photodiode, or the like), etc.

In some other cases, the trackable structure 130 is implemented to be included in a hollow tube-like medical device. In the embodiment where the trackable structure 130 is formed in a tube, the tube may be a pure material, a composition, or an alloy. The tube, or any other portion of the trackable structure 130, may comprise metal, rubber, plastic, epoxy, urethane, or some other material. In cases where the tube is pure metal or includes metal in any purity, a generated magnetic field by the rotating permanent magnet 230 within the trackable structure 130 may nevertheless be detectable by a sensor 120. In some cases, the trackable structure 130 is an elongated solid member. In some cases, the trackable structure 130 takes another form.

In FIG. 6A, the trackable structure 130 may be placed through the mouth of the patient 140 or through another of the patient's orifices. (The method of inputting the structure 130 into the body is not shown in FIG. 6A, and any acceptable method as is known in the art can be used.) Alternatively, the trackable structure 130 may be placed through a surgical incision made by a medical practitioner at some location on the body of the patient 140. The trackable structure 130 may be placed and moved in other ways.

A magnetic field sensing device included in the sensor 120 is operated by a medical practitioner proximal to the body of the patient 140. In some cases, the medical practitioner places the magnetic field sensing device 120 directly in contact with the body of the patient 140. Generally speaking, the medical practitioner will attempt to place the magnetic field sensing device 120 adjacent to the portion of the body where the trackable structure 130 is believed to be.

A presentation system, which may be along the lines of the monitoring station 125 (FIG. 1) includes one or more of a video display, an audio input/output system, a tactile feedback system, or some other presentation mechanism. The presentation system 125 may further include one or more user input interfaces for keyboards, touch screens, mouses, pads, buttons, dials, and other like controls. In some embodiments, the presentation system 125 provides input information to the magnetic field sensing device 120 and receives output information from the magnetic field sensing device 120. Embodiments of the presentation system 125 are used to present information representing the position and orientation of a trackable structure 130 by receiving and processing the harmonics generated by the magnetic field information provided by a low-frequency rotating permanent magnet.

In some embodiments, the magnetic field sensing device 120 includes an electrical conduit 112. The sensing device 120 is shown in FIG. 6A for perpendicular to the patient for ease of understanding in the device, but as can be appreciated, it use it will often be parallel positioned to the patient's body as shown in FIGS. 1 and 6B. The electrical conduit 112 may be used to pass power signals, control signals, data signals, or some other type of electrical signals. In the embodiment of FIG. 6A, the electrical conduit 112 is arranged to pass electrical signaling information to the low-frequency permanent magnet structure 230. The electrical conduit 112 may pass electrical signals in a point-to-point arrangement, serial arrangement, parallel arrangement, networked arrangement, and alternatively, in some other arrangement.

The electrical conduit 112 may be used to pass signaling information between the magnetic field sensing device 120 and the presentation system 125. The electrical conduit 112 may in addition or, in the alternative, pass signaling information between the magnetic field sensing device 120 and the low-frequency magnet structure 130. The signaling information may include power signals, control signals, data signals, or other signals.

In some embodiments, the magnetic field sensing device 120 may include one or more wireless transceivers arranged to communicate data between the magnetic field sensing device 120 and the presentation system 125. In these and other embodiments, the magnetic field sensing device 120 may include one or more wireless transceivers arranged to wirelessly communicate information (e.g., information to generate a particular excitation signal) between the magnetic field sensing device 120 and the low-frequency permanent magnet structure 230.

FIG. 6B illustrates a low-frequency medical tracking system 600B for detecting a position of a trackable structure 130 within a body of a patient 140, according to at least one embodiment. In FIG. 6B, the system 600B is a low-frequency permanent magnet tracking system. A patient 140 is positioned on a bed (not shown) and receiving medical treatment. The medical instrument including the trackable structure 130 is positioned within the body of the patient 140. A sensor 120 is positioned in proximity to the patient 140. The sensor 120 includes an electrical conduit 134 by which the sensor 120 is electrically coupled to the trackable structure 130 and a display of the monitoring station 125.

The sensor 120 includes a control circuit 108 (FIG. 5) that generates an excitation signal, which is applied to the trackable structure 130 that may be incorporated into a medical instrument. The excitation signal causes a current to flow through a coil (not shown) of the trackable structure 130.

The sensor 120 includes one or more magnetic sensors 114a-114f (FIG. 5). The one or more magnetic sensors 114a-114f are configured to detect the generated magnetic field and to output one or more corresponding sensor signals to the control circuit 108 (FIG. 5). The control circuit 108 analyzes the sensor signals from the one or more magnetic sensors 114a-114f and determines location-based information such as the position, orientation, and motion of the trackable structure 130 within the body of the patient 140.

In at least one embodiment, the control circuit 108 outputs a video signal to the display of the monitoring station 125. The display receives the video signal and displays a representation of the position of the trackable structure 130 within the body of the patient 140. The video signal can include position data indicating position coordinates of the trackable structure 130 within the body of the patient 140. The display of the monitoring station 125 displays the position data so that a medical practitioner, medical personnel, or other technicians can view the position data and the representation of the position of the trackable structure 130 in order to appropriately proceed with the medical procedure.

In at least one embodiment, the system 600B is operated by a medical practitioner. During operation, the medical practitioner positions the sensor 120 closely adjacent to, in direct contact with, or otherwise in proximity of the body of the patient 140. In some embodiments, the medical practitioner will attempt to place the sensor 120 adjacent to a region of the body where the trackable structure 130 is believed to be. Thus, in FIG. 6B, the sensor 120 is shown directly over the trackable structure 130 and in contact or near contact with the body of the patient.

In at least one embodiment, the display of the monitoring station 125 includes one or more of a video display, an audio input/output system, a tactile feedback system, signal lights, or some other presentation mechanism. Though not pictured in FIG. 6B, the system 600B can further include one or more user input systems configured to receive user input via keyboards, touchscreens, mouses, pads, buttons, dials, and other like controls.

In at least one embodiment, the control circuit 108 can output position data to one or more computing systems (e.g., an ultrasound device, a robotic surgical system) that control or manage aspects of the medical procedure. The one or more computing systems can adjust medical equipment in accordance with the position data. Additionally or alternatively, the computing system can output an alert indicating to medical personnel that there is a potential problem with the position of the trackable structure 130 within the body of the patient 140.

In some embodiments, the electrical conduit 134 may be used to pass power signals, control signals, data signals, or other types of electrical signals. The electrical conduit 134 may be arranged to pass electrical signaling information to the trackable structure 130 disposed within the medical instrument. The electrical conduit 134 may pass electrical signals in a point-to-point arrangement, a serial arrangement, a parallel arrangement, a network arrangement, and/or in some other suitable arrangement. In some embodiments, the electrical conduit 134 is comprised of wired means such as solid or stranded copper-based wire, wireless means such as a point-to-point or other wireless transceiver, or a combination of wired and wireless means. In case of utilizing wireless means, the embodiments may be substantially identical to the embodiments explained in connection FIG. 6A.

The electrical conduit 134 may be used to pass signaling information between the sensor 120 and the display. Additionally or alternatively, the electrical conduit 134 may pass information between the sensor 120 and the trackable structure 130. The signaling information may include power signals, control signals, data signals, or other signals.

In one or more embodiments, the sensor 120 may include one or more wireless transceivers arranged to communicate data between various components of the system 600B described herein. For example, data or other signals may be wirelessly communicated between any or all of the sensor 120, the display of the monitoring station 125, the control circuit 108, the control unit 310, the trackable structure 130, and other electronic systems that cooperate with these devices such as monitoring equipment, medical diagnostic equipment, and the like. In these and other embodiments, the sensor 120 may include one or more wireless transceivers arranged to communicate data between the components including the sensor 120 and the trackable structure 130.

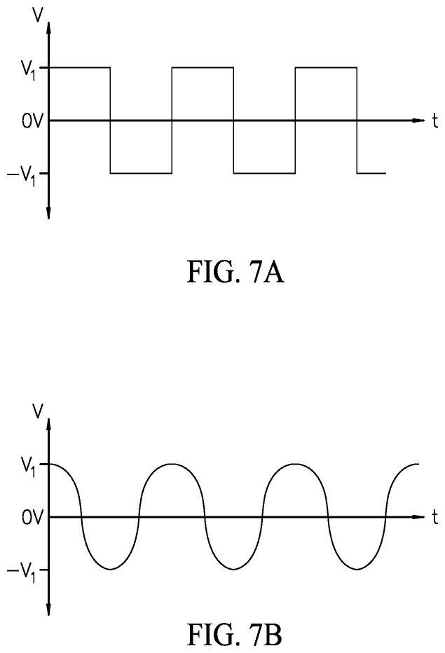

FIG. 7A is a graph of a square wave excitation signal that can be applied to a coil of the trackable structure, according to one or more embodiments. FIG. 7B is a graph of a sine wave excitation signal that can be applied to a coil of the trackable structure, according to one or more embodiments.

In FIG. 7A, the excitation signal is a square wave that oscillates between Vi and −Vi. In the graph of FIG. 7B, the excitation signal is a sinusoidal voltage that oscillates between Vi and −Vi. In some cases, Vi is a voltage of 2.5 volts, 5 volts, or another value. In some cases, −Vi is a voltage of −2.5 volts, −5 volts, or another value. In FIGS. 7A and 7B, the reference voltage between Vi and −Vi is represented as zero volts, 0V. It is recognized, however, that the reference voltage of other embodiments may be above zero volts or below zero volts. In addition, it is further recognized that the absolute value of Vi and −Vi may be a different value. That is, in some embodiments, Vi may be three volts and −Vi may be −2 volts. Other different voltage values are also contemplated.

The excitation signals of FIGS. 7A and 7B are low-frequency excitation signal embodiments, which may be applied to opposing ends of a conductor of a coil 220. The excitation signals of FIGS. 7A and 7B may have a frequency of about 300 Hz, 330 Hz, 500 Hz, 700 Hz or another frequency below about 2,500 Hz.

In some cases, the excitation signals of FIGS. 7A and 7B are pulses in a particular pattern, for example an excitation signal identifier code, as opposed to a constant frequency. Pulses of the excitation signal in these embodiments may be phase shifted to modulate an identifiable code through a generated magnetic field. One excitation signal may have a different duty cycle than another excitation signal. For example, the excitation signal illustrated in FIG. 7B has a lower duty cycle than the excitation signal illustrated in FIG. 7A. Reducing a duty cycle may lower the operating temperature of a particular trackable structure 130 or provide other beneficial characteristics (such as less heat dissipation, lower energy requirement, or the like).

In some embodiments, the magnet drive signals may be generated using a pulse-width modulation (PWM). For example, a pulse width modulated signal with more than 2 states can be used. For instance, a square wave with a positive state, a zero (0) state, and a negative state may be utilized. In further embodiments, a PWM approximation of a sine wave may be used. For instance, a sine wave that has a high frequency modulation between 0 and 1, or 0 and −1 may be utilized.

The present disclosure describes a spinning magnet. The spinning magnet may feel forces from other magnets and other magnetizable materials. These forces will be in some direction and with some torque. One possible impact from external forces, for example, is that the external force or field exerts significant torque such that the permanent magnet rotor drive cannot overcome it. That is, the field from the external source is on the same order as the field generated by the drive coils 220. This will not normally occur, but if this occurs, the necessary force from the drive coils 220 is reduced if the permanent magnet rotor 230 is already spinning because the torque from the external force is conservative (e.g., it will speed up the rotor as well as slowing it down over the course of a full cycle). If there is enough energy already present in the rotation to overcome this energy, then the spinning of the permanent magnet 230 may continue. Other possible impact to consider is from magnetizable materials that are asymmetric about the rotor. That is, the rotor may induce magnetism in them. This magnetism may act to exert a torque on the rotor towards a particular (least energy) alignment. Much like the torque from an external field generator, the impact is reduced if already spinning. Dissipation of induced fields in nearby conductors and magnetic materials implies energy lost and implies drag on the rotor. The drive fields by the coils 220 must be able to overcome this drag. In some embodiments, magnetizable materials may naturally exert a force on the rotor pulling it towards them. This will result in greater force on the bearings, resulting in more drag. The slower the rotor is spinning, the greater this force is likely to be, as the fields would penetrate further into the magnetizable material. Accordingly, in some embodiments, the degree of the excitation signal applied to the drive coils 220 may be set to address the possible issues considered above.

In some embodiments, it is preferable to drive the transverse field coils with a known frequency (synchronous).

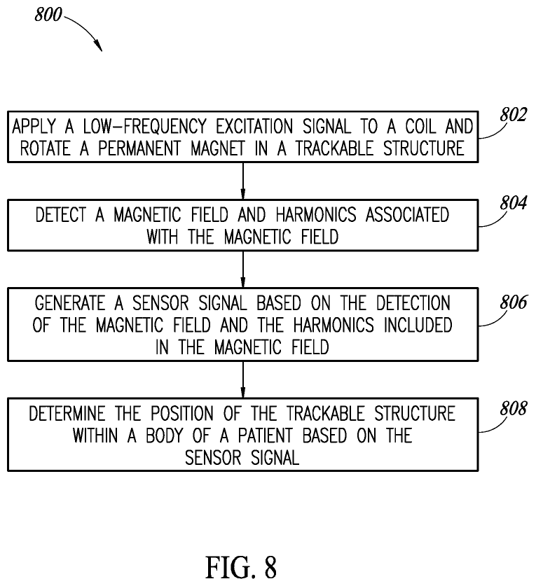

FIG. 8 is a flowchart of a method 800 for detecting the position of a trackable structure 130 within a body of a patient 140, according to one or more embodiments of the present disclosure.

At 802, a low-frequency excitation signal is applied to the coil 220 and the permanent magnet 230 in the trackable structure 130 rotates.

At 804, a sensor 120 senses the magnetic field and the harmonics associated with the magnetic field.

At 806, the sensor 120 generates a sensor signal based on the detection of the magnetic field and the harmonics included in the magnetic field.

At 808, the control unit 310 determines the position of the trackable structure 130 within the body of the patient 140 based on the sensor signal.

The present disclosure provides a method of tracking a spinning permanent magnet with a 6 DOF. When the material bulk (such as the permanent magnet 230) rotates, it is possible to overcome magnetic pinning forces. This has the effect of unpinning the pinning materials. The pinning materials are beneficial to prevent disorder (e.g., low magnetization state) due to self-fields. However, with the rotation of the permanent magnet, the magnetic pinning forces are then used, in part, for maintaining consistent magnetization throughout the bulk material of the permanent magnet.

For example, with high magnetic coercivity materials, it is possible to magnetize a cylinder in the transverse (to axis of symmetry) direction. The cylinder shaped permanent magnet is then set into rotation through the use of transverse field coils applying a torque. The fields from these drive coils (e.g., coils 220) are relatively small compared to the field from the spinning magnet. Effectively, slowly rotating the magnetization vector with a minimum of resisting forces is possible.

In some cases, dissipation due to the induced currents in any nearby conductors is expected. For example, the device might be used in an endoscope of any type, such as a colonoscope, laparoscope, bronchoscope, or the like made of a metal frame. It is likely that this may produce forces if the conductors are close enough. Similar issues apply with nearby magnetic materials as the dimensions involved may result in larger regions of high magnetic field. Nearby magnetic materials could exert significant forces on the high coercivity core, resulting in higher friction and/or torques applied to the core. In some cases, these external factors may modulate the rate of rotation but not to a significant level.

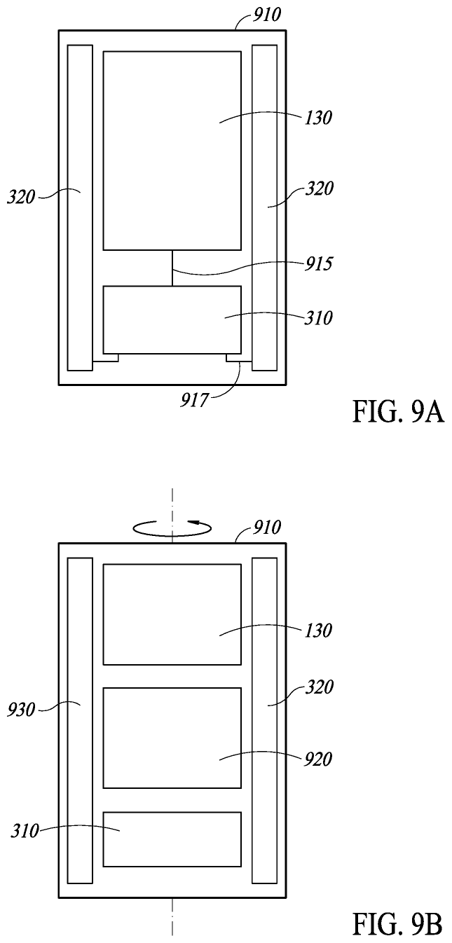

In these cases, the strongest signal is expected to be at a relatively pure frequency. Certain imperfections and designed-in choices may lead to harmonics. In the present disclosure, these harmonics are intentionally used to obtain 6 DOF by signaling the phase of rotation that corresponds with a phase of magnetic field. These harmonics may be ‘naturally’ present due to the number of coils activated for a full revolution. They may be generated such that they are strongest at one particular phase of rotation. These harmonics may also be generated by placing a magnetically reactive material 930 at a given phase of rotation (e.g., chiefly populating the 2f harmonic) adjacent to the permanent magnet 230. (See FIG. 9B) In some embodiments, the permanent magnet structure can be constructed as a permanent magnet assembly. For such embodiments, the two magnet components, the permanent magnet and the reactive magnet material 930 can be considered together as the permanent magnet assembly. In other embodiments, the permanent magnet alone can be considered the permanent magnet assembly. The reactive magnet material 930 can be a metal with a magnetic response, such as iron, nickel, yttrium, neodymium or the like that is not magnetized. Alternatively, it can be a magnetic material that has been magnetized with a magnetic field that interacts with the permanent magnet's magnetic field.

It may be useful to reduce or minimize mechanical energy losses as much as possible; any reduction results in less required power, battery pack volume, and heat generation. This may take the form of unique bearing designs. In some cases, it may be designed to pump the atmosphere in such a fashion (when rotating) as to create an effective air bearing. In some cases, it may be possible to make the bearing magnetically levitated. It may also take the form of altering the atmosphere surrounding the rotor. For example, making the rotor spin in a vacuum, partial vacuum, light gasses, or some combination thereof. Further, in some cases, it may be beneficial to reduce or minimize the static friction. The physics of the dynamic losses is such that higher frequencies rapidly require more energy. Solid on solid losses may scale linearly with rotational frequency (not counting vibration). For example, losses from interactions with the atmosphere should scale as frequency\hat{ }² (e.g., windage, special infinite effective length aerodynamic case).

In one embodiment, the minimization equations is modified to cope with a rotating magnetic field vector (and solving for the spin axis). Relative phase information and amplitude information may be used to solve the equation mentioned above at the primary rotation frequency. One example is, synchronous sampling (e.g., sampling at 2x spin frequency) that may yield a magnetic field of the rotating permanent magnet. In particular, a snapshot may be taken in time of the orientation of the magnetic fields in one direction and then the opposite direction. This may be used to solve for location and get a direction transverse to the spin axis. If additionally sampling in between those times (summing to a second magnetic field set) is performed, obtaining magnetic fields displaced 90 degrees in phase is possible, and hence, after solving for orientation, a direction 90 degrees rotated by the spin axis. The two directions (cross product) together may yield the spin axis (and the correct orientation). In other embodiments, additional calibration (for phase, such as frequency calibration) associated with the sensors may be considered.

The way of tracking the rotating magnetic material with 6 DOF is further described.

Modulating the effective magnet strength as a function of rotation may yield a way to detect rotor phase=0 (possibly with some sign ambiguity). However, if the magnet is considered to be symmetric about its spin axis and has a uniform magnetization that is perpendicular to that spin axis, then the detected signal may be a substantially pure sinusoid because of the linear relationship between field direction of each finite element source and the field detected at the sensor 120. The distances for each finite element may all be the same (this may normally give rise to the non-linear response), removing the potential for non-linearity. Accordingly, the magnitude of field at each sensor should be constant with phase.

The field magnitude along the surface of the cylinder may vary depending on whether a pole is directly adjacent. A reactive element, along some critical axis (phase aligned), may react to this field to become magnetized in the same or opposite direction and may add (or subtract) from the field detected at the sensors. Further, this reactive element may be shape dependent. For example, being relatively long and skinny may enhance its ability to be magnetized in some directions and retard it in others. Accordingly, in some embodiments of the present disclosure, it is beneficial for the permanent magnet to have a mirror symmetric (such that its mirror plane includes the spinning axis) so that minimal shift in solved position occurs. In some embodiments, it may be located outside the stator (to maintain maximum torque on rotor). In some embodiments, it may be composed of ferrite (to reduce or minimize induced current losses). This method may provide two signal peaks per cycle and here, it may be possible to bias the material with a stationary permanent magnet to allow the gain to be higher for one adjacent pole than the other.

In other embodiments, pulse width modulation (PWM) of the stator drive signal and detecting this pattern at the sensors 120 may be used to detect the 6 DOF of the permanent magnet. This involves modulating the drive signal at a different frequency for different drive phases. It may require the detection and decoding of this very small (and higher frequency) drive signal at the sensors.

FIG. 9A is a diagram illustrating a rotating trackable structure, according to one or more embodiments. In the figure, a magnetic trackable assembly or a trackable structure 130 is connected to a control unit 310 and a power source 320. The magnetic trackable structure 130 may be implemented as those shown in connection to FIGS. 2B, 2C, 2D. An outer housing 910 may further house the magnetic trackable structure 130, the control unit 310, and the power source 320. Electrical connection 915 is provided between the magnetic trackable structure 130, and the control unit 310 that permits transfer of data, instructions and, if needed electrical power and other signals. Electrical connection 917 provides power from the power source 320 to the control unit 310. The housing materials used for the housing 910 of FIG. 9A may be substantially identical to the materials used for the housing 210 explained in connection to FIGS. 2B, 2C, 2D.

In some embodiments, the control unit 310 described in connection with FIG. 9A can be implemented in a same or similar manner as the control unit 310 described in connection with FIG. 3. The control unit 310 can therefore be inserted inside of the patient. In some embodiments, in the interest of providing a form factor capable of being inserted inside a patient, some components of the control unit 310 explained in connection with FIG. 3 may be omitted. The control unit 310 of FIG. 9A may include microprocessors, microcontrollers, integrated circuits (ICs) or the like. The one or more microcontrollers may control the coils locally. In some embodiments, the microcontrollers 310 transmit phase information to an external processor for processing and integration with sensor data.

The rotating trackable structure 130 which may implemented as the permanent magnet assembly in some embodiments, may include a microcontroller for controlling the signals driving the rotation of the permanent magnet. Within the permanent magnet assembly, a power source for driving the rotation of the permanent magnet may be included.

In some embodiments, the permanent magnet assembly is connected to the sensor 120 to synchronize the rotation with the sensor 120.

In some embodiments, the permanent magnet assembly may have an additional counter-rotating structure adjacent to the permanent magnet assembly. These arrangements also generate a plurality of harmonic signals.

The power source 320 may include batteries that have a form factor capable of being inserted inside a patient.

FIG. 9B is a diagram illustrating a rotating trackable structure, according to another embodiment of the present disclosure.

A housing 910 may include within a magnetic trackable structure 130, a second rotating structure 920, a control unit 310, a power source 320, and a miscellaneous component section 930. In some embodiments, the second rotating structure 920 may be substantially identical to the magnetic trackable structure 130. In some embodiments, the rotational rate and/or the rotational direction of the magnetic trackable structure 130 and the second rotating structure 920 may be identical to each other. However, in other embodiments, the rotational rate and/or the rotational direction of the trackable structure 130 and the second rotating structure 920 may be different from each other. For example, the trackable structure 130 and the second rotating structure 920 may be identical magnetic structures that rotate in an opposite direction from each other at either a same rotation rate or a different rotation rate. That is, the trackable structure 130 and the second rotating structure 920 may be counter rotating structures with respect to each other, and these counter rotating structures be utilized in part for countering total rotational inertia and also to improve the sensitivity of the apparatus. In some embodiments, the counter rotating structure which is the second rotating structure 920 may also provide a more balanced state during rotation. The counter rotating structure may also help in reducing vibration during rotation.

In some embodiments, the second rotating structure 920 may be non-magnetic.

In some embodiments, the miscellaneous component section 930 may include a battery like the power source 320. In some embodiments, the miscellaneous component section 930 may include sensors such as gyroscopes. In some embodiments, the miscellaneous component section 930 may include reactive magnetic elements. In other embodiments, the miscellaneous component section 930 may include both sensors and reactive magnetic elements. In further embodiments, the miscellaneous component section 930 may include other suitable structures capable of tracking the magnetic structure with at least 5 DOF (thus including 6 DOF). Other embodiments, such as those shown in FIGS. 2B-2G may also include reactive magnetic elements adjacent to, but spaced a small distance away from, the rotating magnet 230.

In some embodiments, the miscellaneous component section 930 may include a device or a module for monitoring the phase of the rotating permanent magnet 230 included in the magnetic trackable structure 130. For example, the miscellaneous component section 930 may include inductive pickup, optical transmitter/receiver, or other suitable devices for monitoring the phase of the magnet 230.

As described above, the miscellaneous component section 930 may also include reactive magnetic elements. These reactive element may be positioned adjacent to the trackable structure 130. In some embodiments, the reactive magnetic element may be positioned inside the trackable structure 130 and be positioned adjacent to the rotating magnet 230. These reactive magnetic asymmetry near the rotating magnet 230 are used to generate magnetic harmonics that indicate phase.

In one or more embodiments, to balance the rotational force, torque, weight of the rotating trackable structure, size and weight of the miscellaneous component section 930 may be balanced to that of the power source 320 as well as other components that may be included within the rotating trackable structure.

In yet other embodiments, only the trackable structure 130 and the second rotating structure 920 may rotate inside the outer housing 910 and other components such as the control unit 310, the power source 320 and the miscellaneous component section 930 may not rotate.

In some of the embodiments of FIGS. 9A and 9B, all of the necessary structures to operate the trackable structure 130 can be included within the single housing 910. This housing 910 can include a controller, a battery, coils and other structures. It can communicate outside the body by use of a wireless transmitter and/or receiver. A microprocessor can be included within the controller 310 to manage the rotation of the magnet and the communication to and from the trackable structure 130.

While no electrical connection wires are shown in the FIG. 9B, they can be present in the end structure itself, both within the housing 910 and also going to and from the housing 910 whose place and connection would easily be within the skill of a person of ordinary skill in the art to perform the functions as described herein.

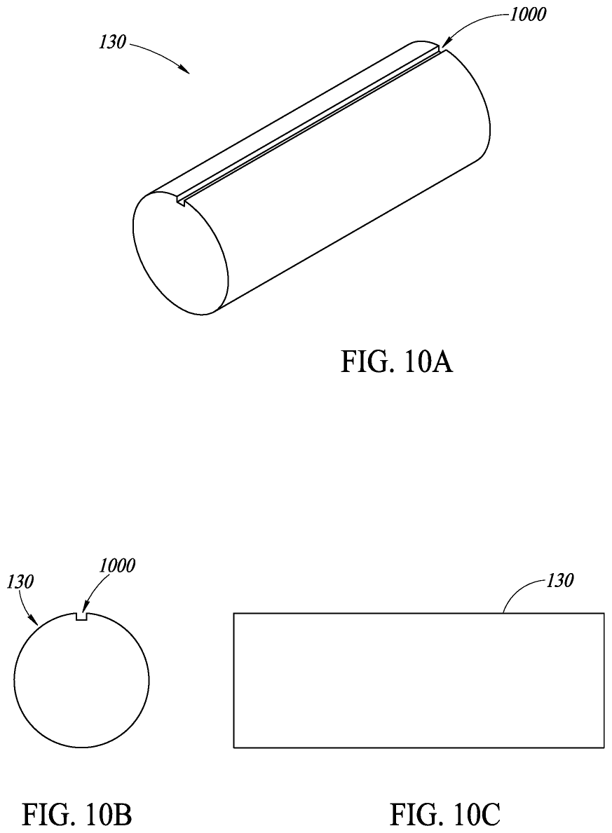

FIG. 10A illustrates a perspective view of a modified housing 210 of a trackable structure 130 according to one embodiment of the present disclosure. FIG. 10B illustrates a front view of a modified housing 210 of a trackable structure 130 according to one embodiment of the present disclosure. FIG. 10C illustrates a side view of a modified housing 210 of a trackable structure 130 according to one embodiment of the present disclosure. These figures relate to a method for mechanical registration of the permanent magnet motor (also referred to as trackable structure 130 in one or more embodiments) according to the present disclosure, and how the trackable structure 130 may be coupled to an external parent device.

In one or more embodiments, the housing 210 of the trackable structure 130 may have modified exterior surfaces to include a registration feature 1000 such as physical registration marks or patterns. The registration feature 1000 allows for lining up the orientation of the stator (e.g., coils 220) with the outside instrument, tracking device, and so forth. The registration feature 1000 on the exterior surfaces of the housing 210 is independent of the rotation and the operation of the permanent magnet 230 itself. However, the registration feature 1000 allows to establish in a fixed position and/or orientation relative to the internal permanent magnet rotor 230. This is beneficial, for example, to know the position of the permanent magnet rotor (with full, 6 degree of freedom) relative to another feature on a medical device of which the permanent magnet rotor may be a part. Further, the registration feature 1000 allows for mechanical registration/positioning of the internal coil structure 220 of the rotor assembly, or the electro-magnetic fields the coils 220 produce, and how the permanent magnet rotor device and its emitted fields may be positioned or coupled to a secondary device.

In certain medical applications and consumer applications, it may be advantageous to integrate the trackable rotor 130 into a secondary device, or device component. Examples of the secondary, medical devices may include endoscopes, catheters, guide wires, needles, drills, screws, rods, or other implants. Example consumer applications may include virtual or augmented reality systems, where the trackable component (rotor) may be integrated in joysticks, controllers, or other devices. Some of these tools/components may have polarizing features or non-aligned shapes (e.g., they may be bent, curved, or have some sort of other protruding feature related to their function).

Generally, in order to track/navigate a device, or a specific feature of a device like the proposed rotor according to the present disclosure (in 6 DOF), calibration of the device orientation (relative to the sensor) prior to use may be required. However, the utilization of the registration feature 1000 provides an approach to eliminate the need for some or all aspects of calibration of the tool/device with this type of tracking system. One method of modifying the outer housing 210 of the rotor assembly 130 by adding a polarizing or registration features 1000 (such as a keyway) are shown in connection with FIGS. 10A, 10B, 10C. While FIGS. 10A, 10B, 10C shows the keyway as one example of the registration feature 1000, various shapes, patterns of physical registration marks may be utilized. With various types of registration features 1000, the orientation of the trackable rotor structure's magnetic fields would be characterized or positioned (i.e., registered) in a known fashion relative to the mechanical features of the housing (e.g., the keyway).

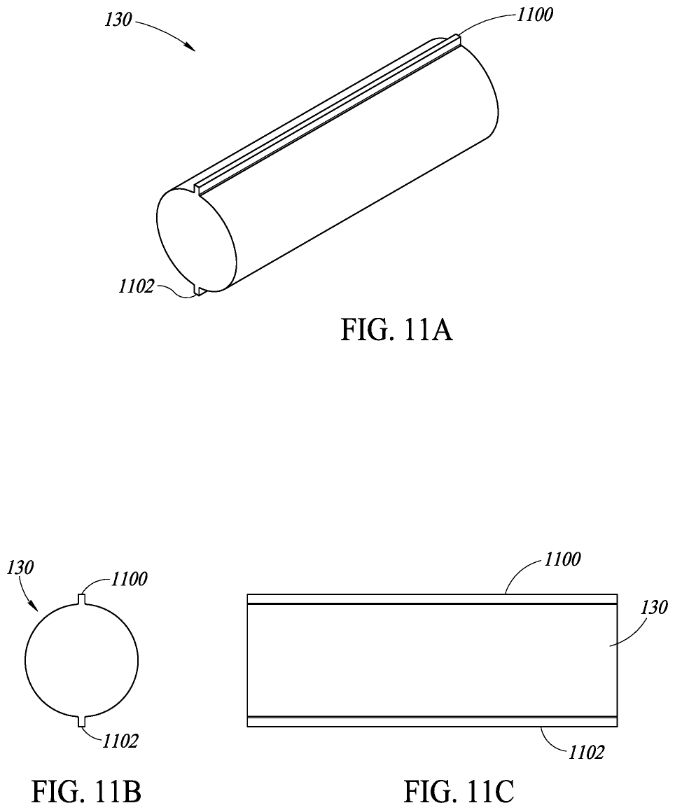

FIG. 11A illustrates a perspective view of a modified housing 210 of a trackable structure 130 according to another embodiment of the present disclosure. FIG. 11B illustrates a front view of a modified housing 210 of a trackable structure 130 according to another embodiment of the present disclosure. FIG. 11C illustrates a side view of a modified housing 210 of a trackable structure 130 according to another embodiment of the present disclosure. These figures relate to another method for mechanical registration of the permanent magnet rotor according to the present disclosure, and how the trackable structure 130 may be coupled to an external parent device.

In one or more embodiments, the housing 210 of the trackable structure 130 may have modified exterior surfaces that includes protruded tabs. A first tab 1100 is located on an exterior surface of the housing 210 and a second tab 1102 opposite of the first tab 1100 is also located on the exterior surface of the housing 210. In some embodiments, the first and second tabs 1100, 1102 may be part of the housing 210 that is protruded from the exterior surface of the housing 210. In other embodiments, the first and second tabs 1100, 1102 may be a separate structure from the housing 210 that is movably attached to the exterior surface of the housing 210. These tabs, which are also an example of the registration feature 1000, allows for lining up the orientation of the stator with the external instrument. Further, the orientation of the rotor's magnetic fields would be characterized or positioned in a known fashion relative to the double tabs of the housing.

For both methods described in FIGS. 10A-10C and 11A-11C, the structural registration features (e.g., keyway, tabs) of the housing in the rotor assembly 130 would mate, or otherwise pair with corresponding structural features in a secondary device such that a mechanical correlation between the secondary device and the magnetic field(s) of the rotor 230 could easily be established.

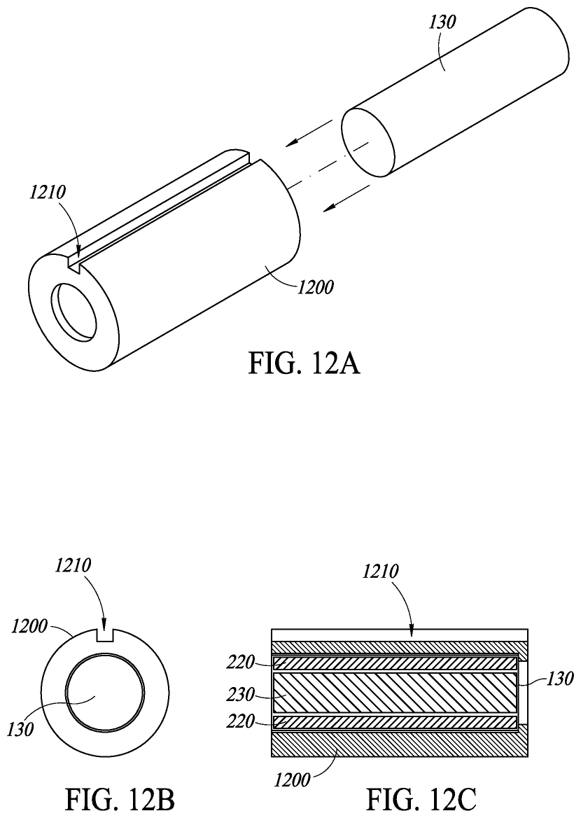

FIG. 12A illustrates a perspective view of a secondary housing 1200 for housing a trackable structure 130 according to one embodiment of the present disclosure. FIG. 12B illustrates a front view of a secondary housing 1200 for housing a trackable structure 130 according to one embodiment of the present disclosure. FIG. 12C illustrates one cross-sectional view of a secondary housing 1200 for housing a trackable structure 130 according to one embodiment of the present disclosure. These figures relate to a secondary housing 1200 with registration features 1210 for housing the motor assembly 130.

In one or more embodiments, the secondary housing 1200 of the trackable structure 130 may have modified exterior surfaces to include a registration feature 1210 such as physical registration marks or patterns. As shown in FIGS. 12A, 12B, 12C the registration feature 1210 includes a keyway that is formed on the outer surfaces of the secondary housing 1200.

The method utilizes the housed motor assembly 130 as described in the present disclosure, bonded to, or assembled with the secondary housing 120 that includes the structural, polarizing/registration feature(s) 1210. The method of application would be similar or identical to that described in connections with FIGS. 10A-10C, 11A-11C. That is, the magnetic fields generated by the rotor assembly 230 would be characterized. The motor assembly 130 is positioned in the secondary housing 1200 (see the direction of shown in FIG. 12A) and the orientation of its magnetic field(s) are registered in a known fashion to the structural registration features 1210 of the secondary housing 1200. Then the motor assembly 130 would be bonded, welded, or otherwise secured to the secondary housing 1200.

In operation, for the methods proposed, the structural registration features 1210 of the secondary housing 1200 would mate, or otherwise pair with corresponding structural features in a secondary device such that a mechanical correlation between the device and the magnetic field(s) of the rotor 230 could easily be established.

A person of ordinary skill in the art would readily understand that while FIGS. 12A, 12B, 12C show the keyway as one example of the registration feature 1210, various physical registration marks with various shapes, sizes, and patterns may be utilized.

In terms of alignment of the motor assembly 130 within the secondary housing 1200, the following calibration methods may be used. For example, for positioning the motor assembly 130 within the secondary housing 1200, a space between the motor assembly 130 and the secondary housing 1200 may be left. Thereafter, the alignment of the motor assembly 130 may be shifted such that the tracking indicates that it is in the proper orientation and position. Once the proper orientation and positions is established, UV cure epoxy may be used to fixate the position.

Another example is to use exterior magnets to align the motor assembly 130 and thus the rotor 230 within the secondary housing 1200 when the rotor assembly 230 is not spinning. Once the proper orientation and positions is established, UV cure epoxy may be used to fix it in place.

Another example for accomplishing axial alignment of both the motor assembly 130 and the rotor 230 within it and the secondary housing 1200 is by placing two copper sleeves at some slight axial distance from the motor assembly 130. The spinning of the rotor assembly 230 then results in a force centering it axially within the secondary housing 1200.

Further approaches for aligning the rotor assembly 130 within the secondary housing 1200 is to activate the rotor assembly 130 within an inclusive copper sleeve. This approach may result in the spin axis aligning with a copper tube axis. In some cases, patterning the copper tube may be beneficial. The aforementioned calibration methods of aligning the rotor assembly 130 within the secondary housing 1200 are examples and a person of ordinary skill in the art would readily appreciate other approaches may be possible. As described throughout herein, the present disclosure describes a novel method for tracking or locating a permanent magnet-based rotor 130 with 6-degree of freedom. The tracking system 300 utilizes a sensor 120 and software algorithm to characterize an alternating magnetic field produced by the rotor's spinning permanent magnet 230. The motor (or the rotor 230 mounted in the trackable structure 130) is placed in the human body and its position and orientation are calculated by the medical tracking system 300. This type of tracking system/rotor may be useful in both medical and consumer markets as previously described.

The present disclosure further provides the following aspects. One aspect of the present disclosure is providing a medical tracking system. The medical tracking system includes: a permanent magnet assembly having a permanent magnet; a conductive wire coil positioned adjacent to the permanent magnet; and a control circuit; and a sensor positioned outside the body of the patient.

In one or more embodiments, the control circuit is configured to apply an excitation signal to the wire coil and cause rotation of the permanent magnet. The permanent magnet generates a magnetic field having harmonics in the magnetic field during the rotation.

The sensor is configured to sense the harmonics of the magnetic field and to output a sensor signal based on the magnetic field that indicates the location of the permanent magnet assembly within the body of the patient based on the sensor signal.

In one or more embodiments, the sensor includes a plurality of first magnetometers configured to detect the magnetic field in a first direction, a plurality of second magnetometers configured to detect the magnetic field in a second direction, and a plurality of third magnetometers configured to detect the magnetic field in a third direction. The first, second, and third directions are all orthogonal to each other.

In one or more embodiments, the sensor signal detected from the plurality of first, second, and third magnetometers includes at least one of an amplitude information, orientation information, phase information, and frequency information generated based on the magnetic field.

The sensor signal includes a first sensor signal detected at a first sensing time and a second sensor signal detected at a second sensing time different from the first sensing time.

In one or more embodiments, the control circuit determines the magnetic field generated by the permanent magnet based on the difference between the first sensor signal and the second sensor signal.

In one or more embodiments, the position information calculated by the control circuit includes a 6 degree of freedom information of the permanent magnet assembly and is based on the difference between the first sensor signal and the second sensor signal.

In one or more embodiments, the permanent magnet assembly includes a magnetically reactive material positioned at a fixed location relative to the permanent magnet.

In one or more embodiments, the position information includes information representing a three-dimensional position of the permanent magnet assembly, an orientation of the permanent magnet assembly, and motion of the permanent magnet assembly with the 6 degree of freedom.

In one or more embodiments, the system further includes a monitoring station. The monitoring station includes a display.

In one or more embodiments, the control circuit is further configured to generate a video signal and to output the video signal to the display of the monitoring station. The video signal includes a representation of the position information.

In one or more embodiments, the permanent magnet assembly further includes a power source for driving the rotation of the permanent magnet.

Another aspect of the present disclosure is providing a medical trackable apparatus configured to be inserted in a body of a patient. The medical trackable apparatus includes: a fixed shaft; a magnetic structure configured to revolve around the fixed shaft at a first rotation rate; coils spaced apart from the magnetic structure at an outer periphery of a permanent magnet; a gap between the magnetic structure and the coils; and a first bearing structure directly contacting the fixed shaft and revolving around the shaft at a second rotation rate. In some embodiments, the gap may be vacuum. In other embodiments, the gap may be filled with gas.

In one or more embodiments, the coils receiving an excitation signal causes the magnetic structure to revolve around the fixed shaft.

In one or more embodiments, the second rotation rate is different from the first rotation rate.

In one or more embodiments, the revolving magnetic structure generates a plurality of trackable harmonics associated with a magnetic field, when the excitation signal is received. For example, one or more trackable harmonics from the revolving magnetic structure may be used for calculating the location, orientation, etc. of the revolving magnetic structure. That is, even one trackable harmonic at one frequency of magnetic field can provide viable tracking.

In one or more embodiments, the magnetic structure includes a permanent magnet.

In one or more embodiments, the medical trackable apparatus further includes a second bearing structure in contact with the first bearing structure. The second bearing structure is placed within a hollow space of the permanent magnet and contacting inner surfaces of the permanent magnet. The first bearing structure includes a plastic washer. The second bearing structure includes a sleeve bearing and rotates at the first rotation rate along the fixed shaft.

In one or more embodiments, the medical trackable apparatus further includes a housing arranged on at least part of the low-frequency trackable structure, and the housing is formed with a bio-compatible material.

In one or more embodiments, the trackable apparatus is configured to be incorporated into a medical instrument.

Yet another aspect of the present disclosure is to provide a method to track a low-frequency trackable structure. The method includes advancing a medical device into a body of a patient. The medical device has a low-frequency trackable structure affixed thereto.

In one or more embodiments, the method includes a step of applying a low-frequency excitation signal to the low-frequency trackable structure.

In one or more embodiments, the method includes a step of rotating the low-frequency trackable structure to generate a magnetic field.

In one or more embodiments, the method includes a step of determining in real time, from outside of the body of the patient. At least one harmonics is associated with the magnetic field produced by the low-frequency trackable structure.

In one or more embodiments, the method includes a step of presenting visual information that tracks motion of the medical device inside the body of the patient based on the detection of the at least one harmonics associated with the magnetic field.

In one or more embodiments, a step of determining at least one harmonics associated with the magnetic field structure includes: a step of detecting the harmonics. The step of determining at least one harmonics associated with the magnetic field structure includes: the step of analyzing the harmonics to obtain at least one of an amplitude information, orientation information, phase information, and frequency information generated based on the magnetic field at a first sensing time and at a second sensing time different from the first sensing time.

The step of determining at least one harmonics associated with the magnetic field structure includes: the step of locating a magnetic dipole based on the difference of the at least one of the amplitude information, orientation information, phase information, and frequency information at the first sensing time and the second sensing time.

The step of determining at least one harmonics associated with the magnetic field structure includes: the step of determining the location of the magnetic dipole as the location of the low-frequency trackable structure.

The various embodiments described above can be combined to provide further embodiments. Further changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A medical tracking system, the medical tracking system comprising:
a permanent magnet assembly having a permanent magnet;
a conductive wire coil positioned adjacent to the permanent magnet; and
a control circuit configured to apply an excitation signal to the wire coil and cause rotation of the permanent magnet, the permanent magnet generating a magnetic field during the rotation; and
a sensor positioned outside the body of the patient, the sensor being configured to sense the magnetic field and to output a sensor signal based on the magnetic field that indicates at least one of location and orientation of the permanent magnet assembly within the body of the patient based on the sensor signal.

2. The system of claim 1, wherein the permanent magnet generates a magnetic field having harmonics in the magnetic field during the rotation, wherein the sensor is configured to sense the harmonics of the magnetic field, the sensor includes a plurality of first magnetometers configured to detect the magnetic field in a first direction, a plurality of second magnetometers configured to detect the magnetic field in a second direction, and a plurality of third magnetometers configured to detect the magnetic field in a third direction, wherein the first, second, and third directions are all orthogonal to each other.

3. The system of claim 2, wherein the sensor signal detected from the plurality of first, second, and third magnetometers includes at least one of an amplitude information, orientation information, phase information, and frequency information generated based on the magnetic field, wherein the sensor signal includes a first sensor signal detected at a first relative phase and a second sensor signal detected at a second relative phase different from the first relative phase.

4. The system of claim 3, wherein the control circuit determines the magnetic field generated by the permanent magnet based on the difference between the first sensor signal and the second sensor signal.

5. The system of claim 3, wherein the position information calculated by the control circuit includes a 6 degree of freedom information of the permanent magnet assembly is based on the difference between the first sensor signal and the second sensor signal.

6. The system of claim 2, wherein permanent magnet assembly includes a magnetically reactive material positioned at a fixed location relative to the permanent magnet.

7. The system of claim 5, wherein the position information includes information representing a three-dimensional position of the permanent magnet assembly, an orientation of the permanent magnet assembly, and motion of the permanent magnet assembly with the 6 degree of freedom.

8. The system of claim 7, wherein the system further comprising a monitoring station including a display, wherein the control circuit is further configured to generate a video signal and to output the video signal to the display of the monitoring station, the video signal including a representation of the position information.

9. The system of claim 1, wherein the permanent magnet assembly further includes a power source for driving the rotation of the permanent magnet and the excitation signal has a frequency below about 2,500 Hz.

10. The system of claim 1, wherein the permanent magnet assembly is connected to the sensor to synchronize rotation with the sensor.

11. The system of claim 1, further comprising a counter-rotating structure adjacent to the permanent magnet assembly.

12. The system of claim 1, wherein the rotation of the permanent magnet assembly generates a plurality of harmonic signals.

13. The system of claim 1, wherein the control circuit receives fundamental rotating frequencies of the rotating permanent magnet sensed through the sensor, and determines position information with a 5 degree of freedom information of the permanent magnet assembly.

14. A medical trackable apparatus configured to be inserted in a body of a patient, comprising:
a fixed shaft;
a magnetic structure configured to revolve around the fixed shaft at a first rotation rate;
coils spaced apart from the magnetic structure, the coils receiving an excitation signal causing the magnetic structure to revolve around the fixed shaft;
a gap between the magnetic structure and the coils; and
a first bearing structure directly contacting the fixed shaft and revolving around the shaft at a second rotation rate,
wherein the revolving magnetic structure generates trackable harmonics associated with a magnetic field, when the excitation signal is received.

15. The medical trackable apparatus of claim 14, wherein the magnetic structure includes a permanent magnet.

16. The medical trackable apparatus of claim 15, wherein the permanent magnet has a tube-like, hollow cylindrical shape.

17. The medical trackable apparatus of claim 16, further comprising a second bearing structure in contact with the first bearing structure,
wherein the second bearing structure is placed within a hollow space of the permanent magnet and contacting inner surfaces of the permanent magnet,
wherein the first bearing structure includes a plastic washer, and
wherein the second bearing structure includes a sleeve bearing and rotates at the first rotation rate along the fixed shaft.

18. The medical trackable apparatus of claim 14, wherein a frequency of the excitation signal is between about 200 Hz and about 700 Hz.

19. The medical trackable apparatus of claim 14, further comprising:
a housing arranged on at least part of the low-frequency trackable structure, the housing formed with a bio-compatible material.

20. The medical trackable apparatus of claim 18, wherein the trackable apparatus is configured to be incorporated into a medical instrument.

21. The medical trackable apparatus of claim 18, further comprising a microcontroller for controlling the excitation signals driving the rotation of the magnetic structure.

22. The medical trackable apparatus of claim 18, further comprising a power source for driving the rotation of the magnetic structure.

23. A method to track a low-frequency trackable structure, comprising:
advancing a medical device into a body of a patient, the medical device having a low-frequency trackable structure affixed thereto;
applying an excitation signal to the low-frequency trackable structure;
rotating the low-frequency trackable structure to generate a changing magnetic field;
determining in real time, from outside of the body of the patient, at least one harmonics associated with the changing magnetic field produced by the low-frequency trackable structure; and
presenting visual information that tracks motion of the medical device inside the body of the patient based on the detection of the at least one harmonics associated with the changing magnetic field.

24. The method of claim 23, wherein determining at least one harmonics associated with the magnetic field structure includes:
detecting the harmonics;
analyzing the harmonics to obtain at least one of an amplitude information, orientation information, phase information, and frequency information generated based on the changing magnetic field at multiple times;
locating a magnetic dipole based on the difference of the at least one of the amplitude information, orientation information, phase information, and frequency information at multiple times; and
determining the location of the magnetic dipole as the location of the low-frequency trackable structure.

25. The method of claim 24, wherein analyzing the harmonics includes using at least one of a Goertzel algorithm or windowed fast Fourier transform.
